(19) United States
(12) Patent Application Publication
(10) Pub. No.: US 2026/0026864 A1
(43) Pub. Date: Jan. 29, 2026

(54) ELECTROSURGICAL INSTRUMENT AND METHOD OF APPLYING ENERGY

(71) Applicant: Cilag GmbH International, Zug (CH)

(72) Inventors: Rachael A. Swenson, Cincinnati, OH (US); Madeleine C. Jayme, Cincinnati, OH (US); Daniel V. Boguszewski, Cincinnati, OH (US); Christopher A. Dums, Neenah, WI (US)

(21) Appl. No.: 18/783,721

(22) Filed: Jul. 25, 2024

(51) Int. Cl. A61B 18/14; A61B 18/00

(52) U.S. Cl. CPC A61B 18/1445; A61B 2018/00619; A61B 2018/0063; A61B 2018/00672; A61B 2018/00678; A61B 2018/00827; A61B 2018/00875; A61B 2018/1455; A61B 2018/1467

(57) ABSTRACT

A surgical system includes electrodes configured to deliver radiofrequency energy to tissue, and a processor configured to control delivery of the RF energy with increasing power, monitor tissue impedance, calculate tissue impedance slope, compare the tissue impedance to a predetermined impedance threshold, and compare the tissue impedance slope to a predetermined impedance slope threshold. The processor is configured to, in response to determining that the tissue impedance is not greater than the predetermined impedance threshold or that the tissue impedance slope is not greater than the predetermined impedance slope threshold, control the delivery of the RF energy to continue with increasing power. The processor is also configured to, in response to determining that the tissue impedance is greater than the predetermined impedance threshold and that the tissue impedance slope is greater than the predetermined impedance slope threshold, control the delivery of the RF energy with a predetermined constant voltage.

### BACKGROUND

A variety of surgical instruments include a tissue cutting element and one or more elements that transmit radio frequency (RF) energy to tissue (e.g., to coagulate or seal the tissue). An example of such an electrosurgical instrument is the ENSEAL® Tissue Sealing Device by Ethicon Endo-Surgery, Inc., of Cincinnati, Ohio. Such electrosurgical instruments may be operated via one or more control algorithms for controlling the delivery of energy to tissue.

In some instances, a control algorithm may determine the exit from the bathtub region (e.g., the time period during energy application where tissue impedance is low enough for electrosurgical energy to be effective for sealing tissue) based on the monitored tissue impedance reaching a first predetermined impedance threshold. Due to variability in tissue impedance, the algorithm may also impose a first minimum time threshold to avoid prematurely detecting exit from the bathtub region, which might otherwise result in a less hemostatic seal. However, imposition of such a minimum time threshold may result in unnecessarily lengthy seal times and/or increased thermal damage, such as in instances where the seal has truly exited the bathtub region prior to reaching the minimum time threshold. Similarly, an algorithm may determine the completion of a sealing cycle based on the monitored tissue impedance reaching a second predetermined impedance threshold and based on the duration of the post-bathtub region of the energy delivery reaching a second minimum time threshold. However, the impedance threshold may be low and thus reached rather quickly, such that the time threshold may ultimately determine the seal cycle termination. As a result, seal cycle times may be the same for most or all tissue types, rather than being based on the actual completion of the seal, which can lead to over-delivery or under-delivery delivery of energy to the tissue.

In some instances, the structure of a control algorithm may not allow the algorithm to switch between different energy output levels without having the energy output level temporarily drop to zero. In addition, the structure of a control algorithm may not be conducive to allowing for tissue classification. In this regard, a control algorithm may have the same power output and logic for all activations, irrespective of tissue type, which may result in a less hemostatic seal, unnecessarily lengthy seal times, and/or increased thermal damage for certain tissue types.

While a variety of surgical instruments have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a perspective view of an exemplary electrosurgical instrument;

FIG. 2 depicts a perspective view of an exemplary articulation assembly and end effector of the electrosurgical instrument of FIG. 1;

FIG. 3 depicts an exploded view of the articulation assembly and end effector of FIG. 2;

FIG. 4 depicts a perspective view of the end effector that of FIG. 2;

FIG. 5 depicts an exploded perspective view of the end effector of FIG. 2;

FIG. 6 depicts an illustrative impedance triangle;

FIG. 7 depicts a set of illustrative example waveforms;

FIG. 8 depicts another set of illustrative example waveforms;

FIG. 9 depicts another illustrative example waveform;

FIG. 10 depicts another illustrative example waveform;

FIG. 11 depicts another set of illustrative example waveforms;

FIG. 12 depicts another illustrative example waveform;

FIG. 13 depicts another illustrative example waveform;

FIG. 14 depicts an illustrative example level of impedance over time present in tissue undergoing a sealing procedure during surgery;

FIG. 15 depicts illustrative example power curves that may be delivered by an electrosurgical system;

FIG. 16 depicts an example block diagram describing the selection and application of composite load curves in a tissue seal control process;

FIG. 17 depicts a flowchart of an example of a method for operating an electrosurgical instrument with detection of bathtub region exit, and with seal completeness sensing;

FIG. 18 depicts an example graph of voltage, current, power, and impedance relative to time when using the method of FIG. 17;

FIG. 19 depicts a flowchart of another example of a method for operating an electrosurgical instrument using lookup tables;

FIG. 20 depicts examples of lookup tables for use in the method of FIG. 19; and

FIG. 21 depicts a flowchart of another example of a method for operating an electrosurgical instrument using automated tissue classification.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

For clarity of disclosure, the terms “proximal” and “distal” are defined herein relative to a surgeon or other operator grasping a surgical instrument having a distal surgical end effector. The term “proximal” refers the position of an element closer to the surgeon or other operator and the term “distal” refers to the position of an element closer to the surgical end effector of the surgical instrument and further away from the surgeon or other operator.

### I. Example of Electrosurgical Instrument

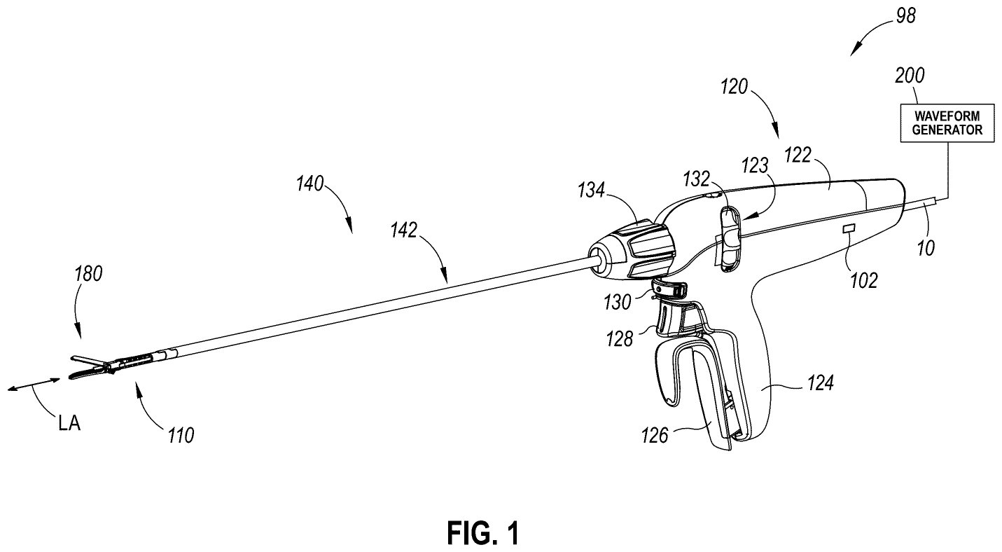

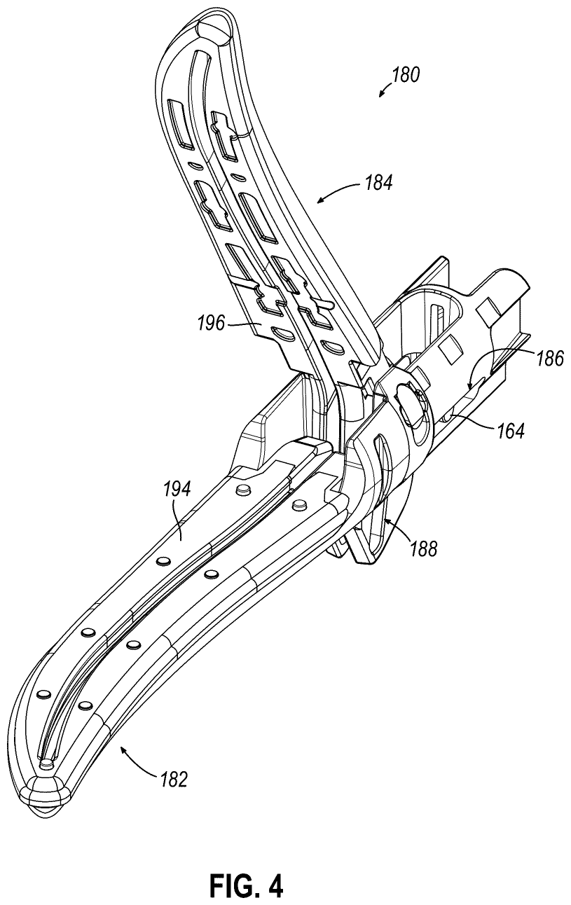

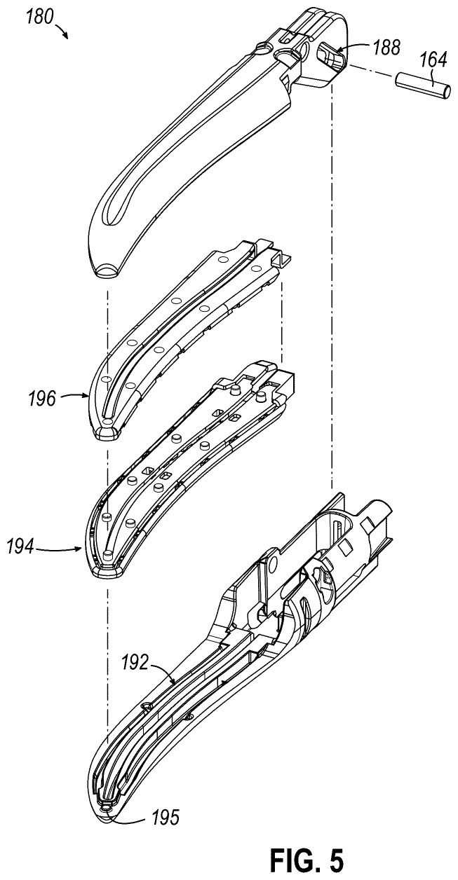

FIGS. 1-5 show a surgical system (98) including an exemplary electrosurgical instrument (100). As best seen in FIG. 1, electrosurgical instrument (100) includes a handle assembly (120), a shaft assembly (140), an articulation assembly (110), which may also be referred to as an articulation section (110), and an end effector (180). As will be described in greater detail below, end effector (180) of electrosurgical instrument (100) is operable to grasp, cut, and seal or weld tissue (e.g., a blood vessel, etc.). In this example, end effector (180) is configured to apply a non-therapeutic bipolar radio frequency (RF) energy in order to identify and/or verify that the correct tissue is present in the end effector such that a therapeutic RF energy can be applied to seal or weld tissue. However, it should be understood that electrosurgical instrument (100) may be configured to seal or weld tissue through any other suitable means that would be apparent to one skilled in the art in view of the teachings herein. For example, electrosurgical instrument (100) may be configured to seal or weld tissue via an ultrasonic blade, staples, etc. In the present example, electrosurgical instrument (100) is electrically coupled to a waveform generator (200) of surgical system (98), which is capable of delivering therapeutic and non-therapeutic energy, via power cable (10).

Waveform generator (200) may be configured to provide all or some of the electrical power requirements for use of electrosurgical instrument (100). Any suitable waveform generator (200) may be used as would be apparent to one skilled in the art in view of the teachings herein. By way of non-limiting example, the waveform generator (200) may be constructed in accordance with at least some of the teachings of U.S. Pat. No. 8,986,302, entitled “Surgical Generator for Ultrasonic and Electrosurgical Devices,” issued Mar. 24, 2015, the disclosure of which is incorporated by reference herein, in its entirety. While in the current example, electrosurgical instrument (100) is coupled to waveform generator (200) via power cable (10), electrosurgical instrument (100) may contain an internal power source or plurality of power sources, such as a battery and/or supercapacitors, to electrically power electrosurgical instrument (100). Of course, any suitable combination of power sources may be utilized to power electrosurgical instrument (100) as would be apparent to one skilled in the art in view of the teaching herein.

Handle assembly (120) is configured to be grasped by an operator with one hand, such that an operator may control and manipulate electrosurgical instrument (100) with a single hand. Although electrosurgical instrument (100) is primarily described herein as being used by a human user, it should be noted that alternative versions exist in which one or more robotic systems (e.g., a robotic arm) may be used to control and manipulate electrosurgical instrument (100). Shaft assembly (140) extends distally from handle assembly (120) and connects to articulation assembly (110). Articulation assembly (110) is also connected to a proximal end of end effector (180). As will be described in greater detail below, components of handle assembly (120) are configured to control end effector (180) such that an operator may grasp, cut, and seal or weld tissue. Articulation assembly (110) is configured to deflect end effector (180) from the longitudinal axis (LA) defined by shaft assembly (140).

Handle assembly (120) of the present example includes a control unit (102) housed within a body (122), a pistol grip (124), a jaw closure trigger (126), a knife trigger (128), an activation button (130), an articulation control (132), and a knob (134). As will be described in greater detail below, jaw closure trigger (126) may be pivoted toward and away from pistol grip (124) and/or body (122) to open and close jaws (182, 184) of end effector (180) to grasp tissue. Additionally, knife trigger (128) may be pivoted toward and away from pistol grip (124) and/or body (122) to actuate a knife member (176) within the confines of jaws (182, 184) to cut tissue captured between jaws (182, 184). Further, activation button (130) may be pressed to apply radio frequency (RF) energy to tissue via electrodes (194, 196) of jaws (182, 184), respectively. In some versions, electrodes (194, 196) of jaws (182, 184) are in a bifurcation configuration where electrodes (194, 196) move relative to a central axis and nearly equal and opposite to one another.

Body (122) of handle assembly (120) defines an opening (123) through which a portion of articulation control (132) protrudes. Articulation control (132) is rotatably disposed within body (122) such that an operator may rotate the portion of articulation control (132) protruding from opening (123) to rotate the portion of articulation control (132) located within body (122). Rotation of articulation control (132) relative to body (122) will bend articulation assembly (110) in order to drive deflection of end effector (180) from the longitudinal axis (LA) defined by shaft assembly (140). Articulation control (132) and articulation assembly (110) may include any suitable features to drive deflection of end effector (180) from the longitudinal axis (LA) defined by shaft assembly (140) as would be apparent to one skilled in the art in view of the teachings herein.

Knob (134) is rotatably disposed on the distal end of body (122) and is configured to rotate end effector (180), articulation assembly (110), and shaft assembly (140) about the longitudinal axis (LA) of shaft assembly (140) relative to handle assembly (120). While in the current example, end effector (180), articulation assembly (110), and shaft assembly (140) are rotated by knob (134), knob (134) may be configured to rotate end effector (180) and articulation assembly (110) relative to selected portions of shaft assembly (140). Knob (134) may include any suitable features to rotate end effector (180), articulation assembly (110), and shaft assembly (140) as would be apparent to one skilled in the art in view of the teachings herein.

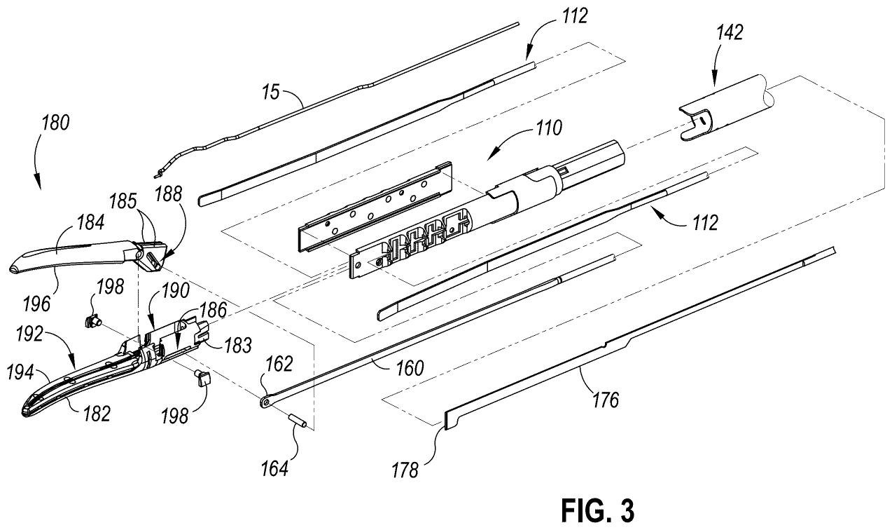

Shaft assembly (140) includes distal portion (142) extending distally from handle assembly (120) and a proximal portion housed within the confines of body (122) of handle assembly (120). Referring to FIG. 3, shaft assembly (140) houses a jaw closure connector (160) that couples jaw closure trigger (126) with end effector (180). Additionally, shaft assembly (140) houses a portion of knife member (176) extending between distal a distal cutting edge (178) of knife member (176) and knife trigger (128). Shaft assembly (140) also houses actuating members (112) that couple articulation assembly (110) with articulation control (132); as well as an electrical coupling (15) that operatively couples electrodes (194, 196) with activation button (130). As will be described in greater detail below, jaw closure connector (160) is configured to translate relative to shaft assembly (140) to open and close jaws (182, 184) of end effector (180); while knife member (176) is coupled to knife trigger (128) of handle assembly (120) to translate distal cutting edge (178) within the confines of end effector (180); and activation button (130) is configured to activate electrodes (194, 196).

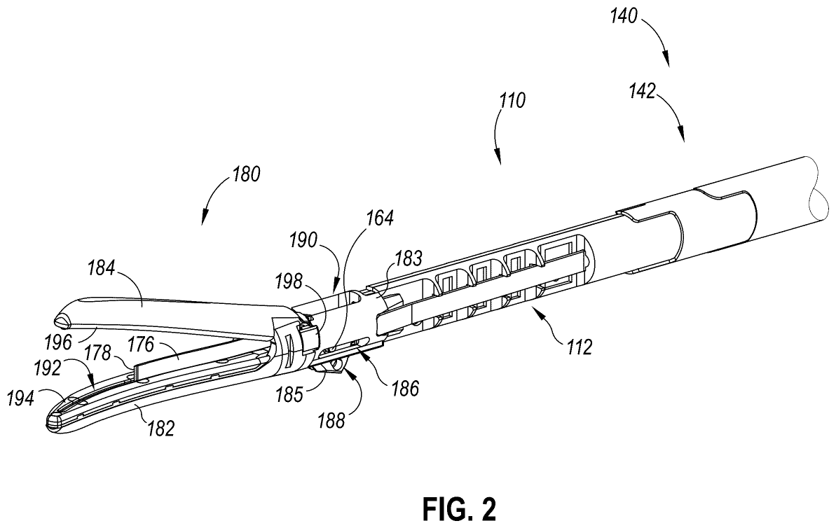

As best seen in FIGS. 2-5, end effector (180) includes lower jaw (182) pivotally coupled with upper jaw (184) via pivot couplings (198). Lower jaw (182) includes a proximal body (183) defining a slot (186), while upper jaw (184) includes proximal arms (185) defining a slot (188). Lower jaw (182) also defines a central channel (190) that is configured to receive proximal arms (185) of upper jaw (184), portions of knife member (176), jaw closure connector (160), and pin (164). Slots (186, 188) each slidably receive pin (164), which is attached to a distal coupling portion (162) of jaw closure connector (160). Additionally, lower jaw (182) includes a force sensor (195) located at a distal tip of lower jaw (182), though force sensor (195) may alternatively be positioned at any other suitable location. Force sensor (195) may be in communication with control unit (102). Force sensor (195) may be configured to measure the closure force generated by pivoting jaws (182, 184) into a closed configuration in accordance with the description herein. Additionally, force sensor (195) may communicate this data to control unit (102). Any suitable components may be used for force sensor (195) as would be apparent to one skilled in art in view of the teachings herein. For example, force sensor (195) may take the form of a strain gauge. In some variations, end effector (180) includes more than one force sensor.

While in the current example, a force sensor (195) is incorporated into electrosurgical instrument (100) and is in communication with control unit (102), any other suitable sensors or feedback mechanisms may be additionally or alternatively incorporated into electrosurgical instrument (100) while in communication with control unit (102) as would be apparent to one skilled in the art in view of the teachings herein. For instance, an articulation sensor or feedback mechanism may be incorporated into electrosurgical instrument (100), where the articulation sensor communicates signals to control unit (102) indicative of the degree end effector 180 is deflected from the longitudinal axis (LA) by articulation control (132) and articulation assembly (110).

As will be described in greater detail below, jaw closure connector (160) is operable to translate within central channel (190) of lower jaw (182). Translation of jaw closure connector (160) drives pin (164). As will also be described in greater detail below, with pin (164) being located within both slots (186, 188), and with slots (186, 188) being angled relative to each other, pin (164) cams against proximal arms (185) to pivot upper jaw (184) toward and away from lower jaw (182) about pivot couplings (198). Therefore, upper jaw (184) is configured to pivot toward and away from lower jaw (182) about pivot couplings (198) to grasp tissue.

The term “pivot” does not necessarily require rotation about a fixed axis and may include rotation about an axis that moves relative to end effector (180). Therefore, the axis at which upper jaw (184) pivots about lower jaw (182) may translate relative to both upper jaw (184) and lower jaw (182). Any suitable translation of the pivot axis may be used as would be apparent to one skilled in the art in view of the teachings herein.

Lower jaw (182) and upper jaw (184) also define a knife pathway (192). Knife pathway (192) is configured to slidably receive knife member (176), such that knife member (176) may be retracted, and advanced, to cut tissue captured between jaws (182, 184).

Lower jaw (182) and upper jaw (184) each comprise a respective electrodes (194, 196). The power source may provide RF energy to electrodes (194, 196) via electrical coupling (15) that extends through handle assembly (120), shaft assembly (140), articulation assembly (110), and electrically couples with one or both of electrodes (194, 196). Electrical coupling (15) may selectively activate electrodes (194, 196) in response to an operator pressing activation button (130). In some instances, control unit (102) may couple electrical coupling (15) with activation button (130), such that control unit (102) activates electrodes (194, 196) in response to operator pressing activation button (130). Control unit (102) may have any suitable components in order to perform suitable functions as would be apparent to one skilled in the art in view of the teachings herein. For instance, control unit (102) may have a processor, memory unit, suitable circuitry, etc. Examples of features and functionalities that may be incorporated into control unit (102) will be described in greater detail below.

As described above, jaw closure trigger (126) may be pivoted toward and away from pistol grip (124) and/or body (122) to open and close jaws (182, 184) of end effector (180) to grasp tissue. In particular, as will be described in greater detail below, pivoting jaw closure trigger (126) toward pistol grip (124) may proximally actuate jaw closure connector (160) and pin (164), which in turn cams against slots (188) of proximal arms (185) of upper jaw (184), thereby rotating upper jaw (184) about pivot couplings (198) toward lower jaw (182) such that jaws (182, 184) achieve a closed configuration.

In some versions, knife trigger (128) may be pivoted toward and away from body (122) and/or pistol grip (124) to actuate knife member (176) within knife pathway (192) of jaws (182, 184) to cut tissue captured between jaws (182, 184). In particular, handle assembly (120) further includes a knife coupling body that is slidably coupled along proximal portion of shaft assembly (140). Knife coupling body is coupled with knife member (176) such that translation of knife coupling body relative to proximal portion of shaft assembly (140) translates knife member (176) relative to shaft assembly (140).

In another version, knife coupling body may be coupled to a knife actuation assembly such that as knife trigger (128) pivots toward body (122) and/or pistol grip (124), knife actuation assembly drives knife coupling body distally, thereby driving knife member (176) distally within knife pathway (192). Because knife coupling body is coupled to knife member (176), knife member (176) translates distally within shaft assembly (140), articulation assembly (110), and within knife pathway (192) of end effector (180). Knife member (176) includes distal cutting edge (178) that is configured to sever tissue captured between jaws (182, 184). Therefore, pivoting knife trigger (128) causes knife member (176) to actuate within knife pathway (192) of end effector (180) to sever tissue captured between jaws (182, 184).

With distal cutting edge (178) of knife member (176) actuated to the advanced position, an operator may press activation button (130) to selectively activate electrodes (194, 196) of jaws (182, 184) to seal or weld severed tissue captured between jaws (182, 184). It should be understood that the operator may also press activation button (130) to selectively activate electrodes (194, 196) of jaws (182, 184) at any suitable time during exemplary use. Therefore, the operator may also press activation button (130) while knife member (176) is retracted. Next, the operator may release jaw closure trigger (126) such that jaws (182, 184) pivot into the opened configuration, releasing tissue.

### II. Sensing Tissue Impedance for Determinations of Tissue State

Electrosurgical instrument (100) discussed above is configured to clamp tissue using end effector (180). Once securely clamped, electrodes (194, 196) in end effector (180) apply a non-therapeutic (i.e., low voltage) waveform to the tissue; and sensor devices measure the returning waveform to calculate and measure the impedance of the tissue. In one example, one or more electrodes (194, 196) are operatively connected to such sensor devices such that electrodes (194, 196) may be referred to as sensors in this respect. More specifically, electrosurgical instrument (100), via one or more sub-circuits, will provide non-therapeutic energy to the extracellular and intracellular fluid present within a given (e.g., clamped) region of tissue to determine a phase and a magnitude of the impedance of the tissue within jaws (182, 184). A processor may then relay information associated with a state of the tissue, such as, for example, tissue type, tissue phase, tissue margin, and the like. Using this associated information, a system inclusive of such electrosurgical instrument (100) cannot only verify that the proper tissue is clamped between jaws (182, 184), but can also determine if any non-tissue material is present between jaws (182, 184), and/or if a proper seal has been created after applying the therapeutic RF energy.

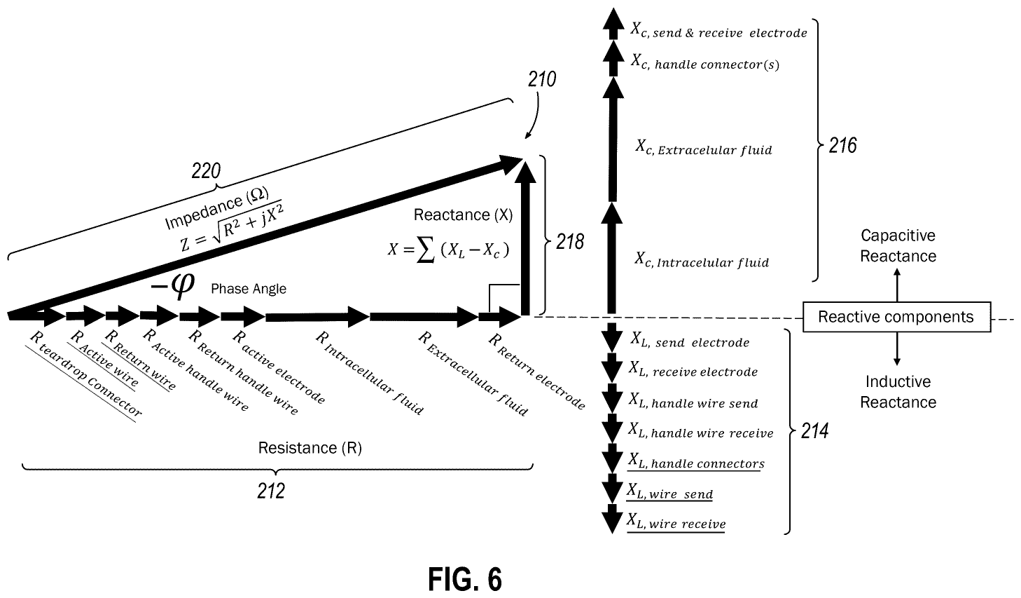

FIG. 6 shows an illustrative impedance triangle (210). As would be understood by one skilled in the art, human tissues may tend to be capacitive in nature, while wires, tool, staples, implants, etc. may tend to be inductive in nature. Thus, as can be seen by the illustrative impendence triangle (210), the “resistance” of each object in the circuit is measured (212) using the waveform and sensor electrodes (194, 196). Such system can also determine the “capacitive reactance” of each object in the circuit and the inductive reactance of each object in the circuit. For example, the send and receive electrodes (194, 196), the send and receive handle wires, the handle connector and the send and receive wires (e.g., included in power cable 10 (see FIG. 1)) all have inductive reactance (214). Additionally, the send and receive electrodes (194, 196), the handle connector, the extracellular fluid, and the intracellular fluid all have capacitive reactance (216). The “reactance” (218) can then be calculated by determining the difference between the capacitive reactance and the inductive reactance using:

$$X = \sum ( {X}_{L} - {X}_{C} ) . \qquad Equation 2$$

As shown in FIG. 6, the “impedance” 220 can then be determined using:

$$Z = \sqrt{{R}^{2} + {jX}^{2}} \qquad Equation 3$$

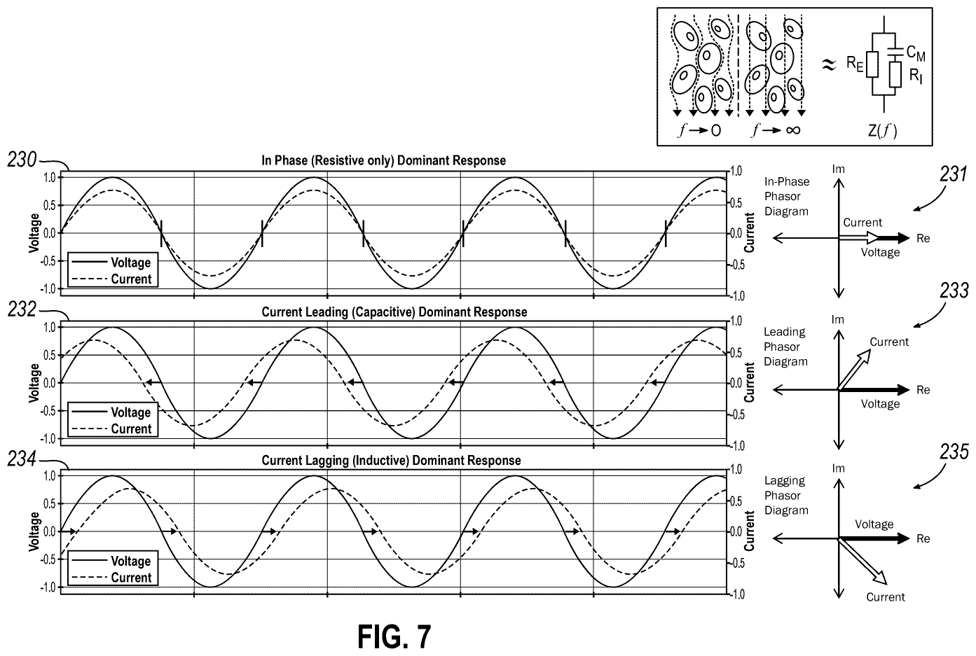

FIG. 7 shows a set of illustrative example waveforms. As would be understood by one skilled in the art, if a circuit only contains resistive items, the current and voltage will remain in phase such as shown in a first graph (230) and a first phasor diagram (231). Alternatively, if the circuit has capacitive objects, or more capacitive than inductive, the voltage wave will lead the current wave such as shown in a second graph (232) and a second phasor diagram (233). Finally, if the circuit has inductive objects, or more inductive objects than capacitive objects, the voltage will lag behind the current, such as shown in a third graph (234) and third phasor diagram (235).

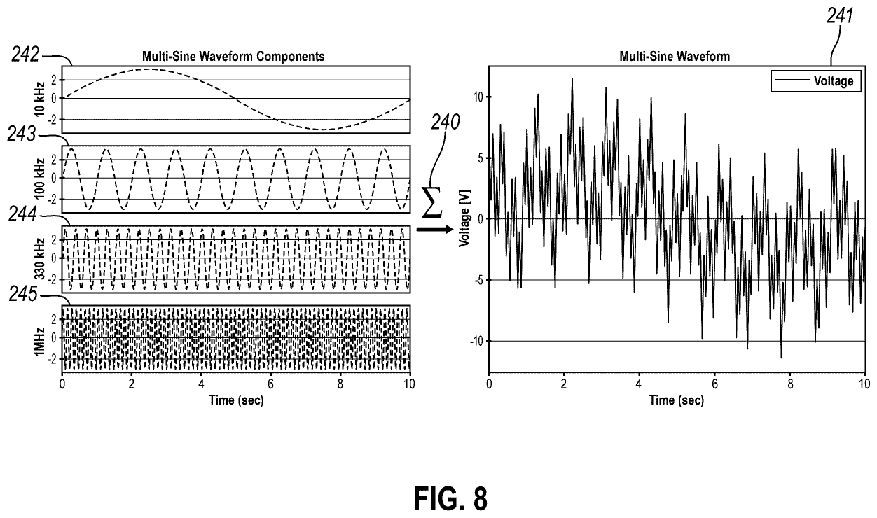

As discussed herein, the system may pass a non-therapeutic waveform through a portion of patient tissue to help identify the type of tissue as well as any foreign objects. Thus, in some versions, the system may pass waveforms of varying frequency (e.g., in series and/or parallel) to improve the accuracy of the determination. Accordingly, in some versions, and as shown in FIG. 8, multiple waveforms of various frequencies may be added or summed together (240) to create a muti-sine waveform (241). By way of non-limiting example, a 10 kHz sine wave (242) may be combined with a 100 kHz sine wave (243), a 330 kHz sine wave (244) and a 1 MHz sine wave (245) may be combined to create multi-sine wave (241).

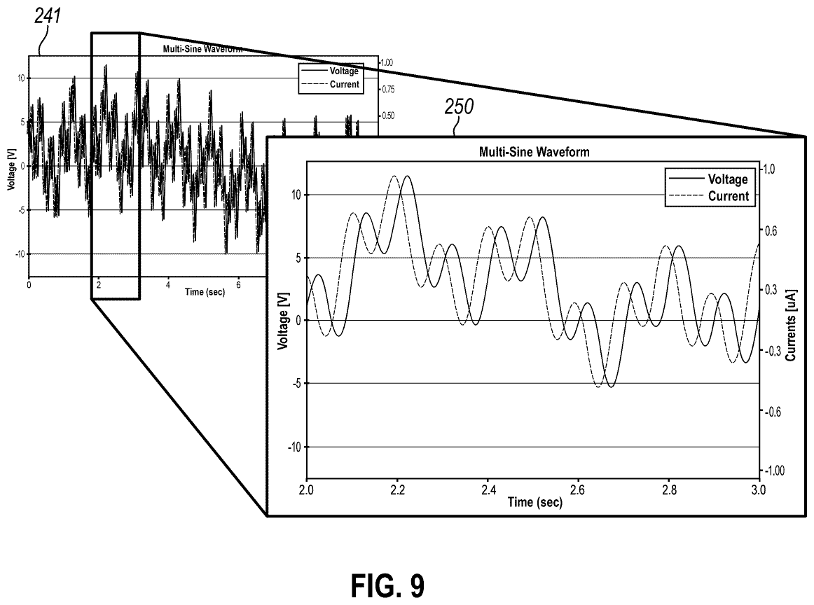

Referring now to FIG. 9, multi-sine waveform (241) may be sampled or windowed. In some versions, such as those that require the use of Fast Fourier Transforms (FFT), the windowing or sampling may be as small as a single period for the lower frequency waveform. As shown in a fourth graph (250), the voltage of multi-sine waveform (241) is leading the current and thus in the present version indicates a capacitive circuit (e.g., likely tissue). In an alternative version, the system may apply a series of burst waveforms having different frequencies.

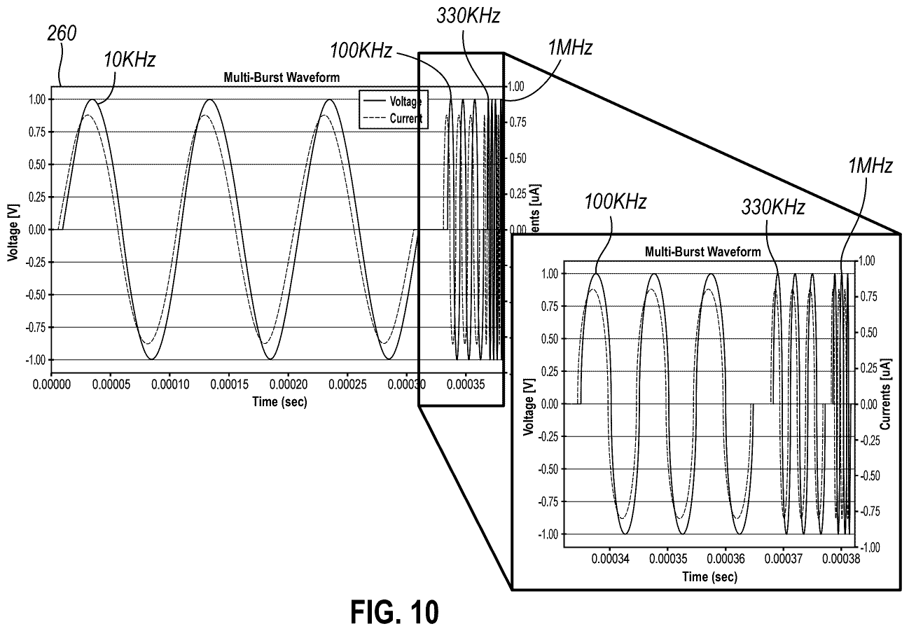

Referring now to FIG. 10, a burst waveform, including a brief delay between frequencies, is shown in fifth graph (260). In some versions, and as shown, the system may output a burst waveform that is a sine wave, while in other versions, the wave may be a square, triangle, ramp, pulse, pseudorandom binary sequence (PRBS), or arbitrary waveform. In some versions, the pause between waveforms can be evaluated in order to determine a “rebounding” time. The rebounding time may be used to help identify tissue types by evaluating how long certain tissues take to allow the waveform and any residual energy to dissipate from the tissue.

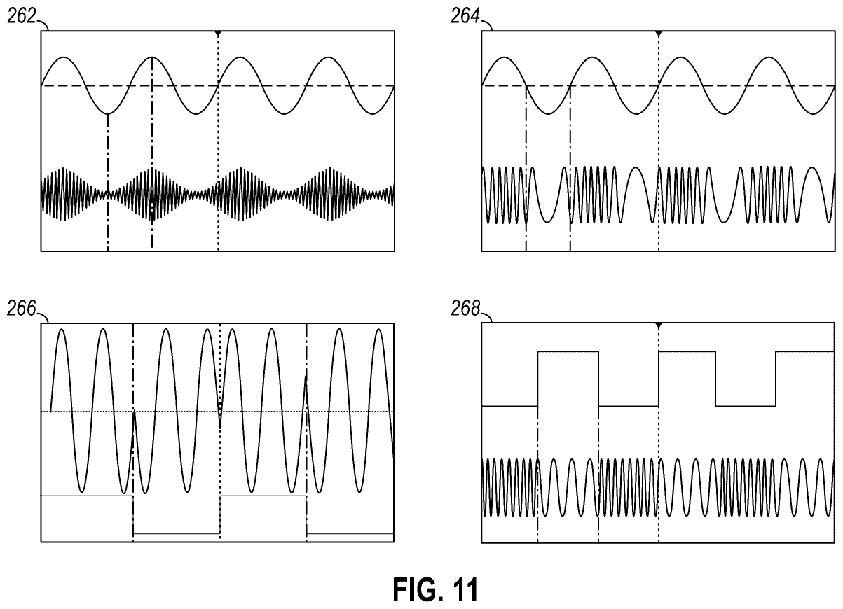

FIG. 11 shows various alternative burst versions. Specifically, in one version, amplitude modulation (AM) (262) may be used; while in another version, frequency modulation (FM) (264). Other versions may use phase modulation (PM) (266) and/or frequency-shift keying (FSK) modulation (268). Due to the fact that all of the modulation options shown in FIG. 11 involve a shift of some type, they may all be evaluated in a similar manner.

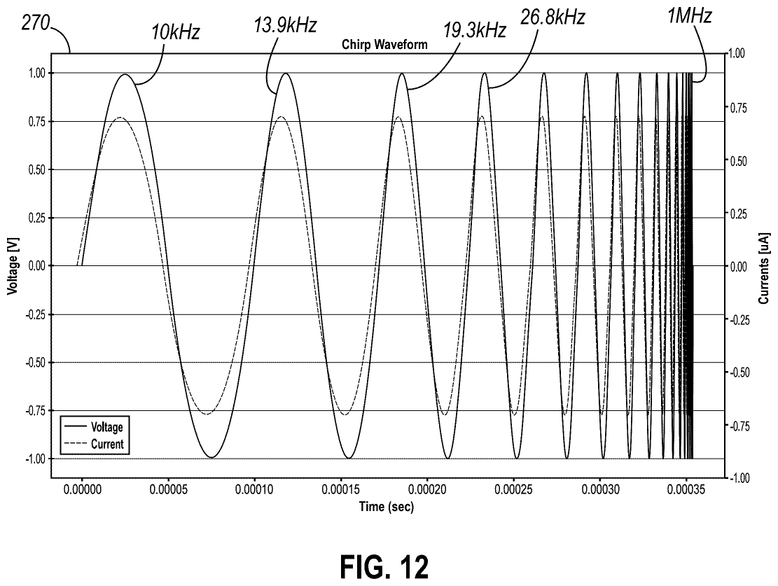

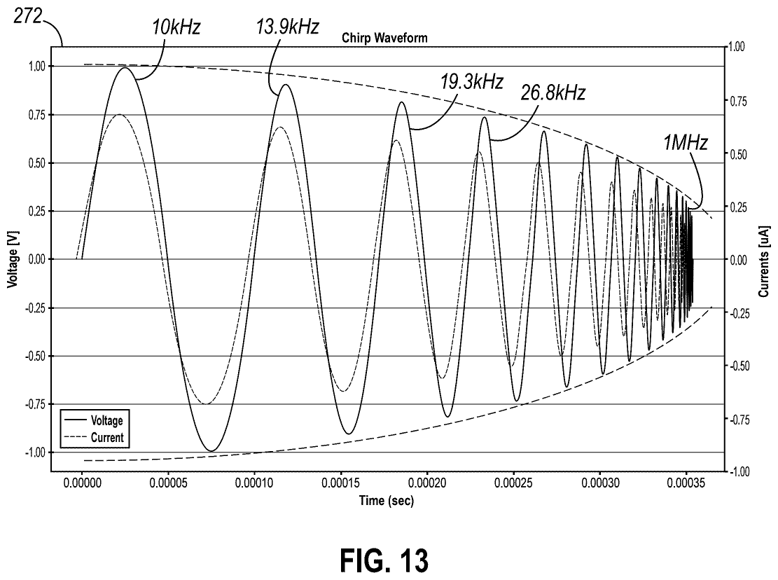

In a further version, a “chirp” function can be used, such as shown in FIG. 12. As would be understood by one skilled in the art, a chirp wave can be an “up-chirp” (i.e., the frequency increases) or a “down-chirp” (i.e., the frequency decreases). Thus, stated differently, a chirp function is essentially an advanced form of FM (264). The chirp function shown in a sixth graph (270) shows a chirp waveform with increasing frequency (e.g., 10 kHz, 13.2 kHz, 19.3 kHz, 26.8 kHz, and 1 Mhz). FIG. 13 shows a seventh graph (272) depicting a chirp function with the same frequencies as shown in FIG. 12, but with a decreasing amplitude. Additional features associated with electrical circuits and measurements of tissue are described in U.S. patent application Ser. No. 17/854,306, entitled “Electrosurgical Instrument for Applying Non-Therapeutic RF Signals,” filed Jun. 30, 2022, and published as U.S. Pat. Pub. No. 2024/0000499 on Jan. 4, 2024, the disclosure of which is incorporated by reference herein, in its entirety.

### III. Bathtub Region

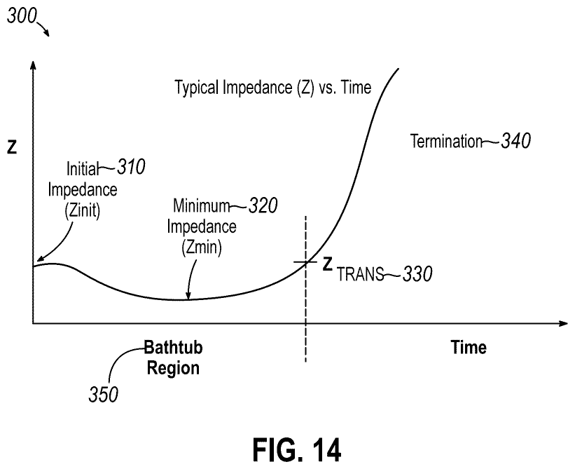

Referring to FIG. 14, graph (300) provides a visual depiction of the level of impedance over time present in tissue undergoing a scaling procedure during surgery. This example graph (300) provides a conceptual framework for the types of power adjustments employed according to the present disclosures. Here, time zero represents the first point at which a surgical instrument, such as instrument (100), applies electrosurgical energy to tissue at a surgical site. The Y axis represents the level of tissue impedance (Z) present when a substantially constant level of power is applied to the tissue via end effector (180) of instrument (100). At time zero, the tissue exhibits an initial level of impedance (Zinit) (310). The initial level of impedance (310) may be based on native physiological properties about the tissue, such as density, amount of moisture, and what type of tissue it is. Over a short period of time, it is known that the level of impedance actually dips slightly as power is continuously applied to the tissue. This is a common phenomenon that occurs in all kinds of tissue. A minimum level of impedance (Zmin) (320) is eventually reached. From here, the overall level of impedance monotonically increases, and at first increases with a slow rise. Eventually, a transition point is reached such that level of impedance starts to dramatically rise above the initial level of impedance (310). This point is generally known as a transition impedance level (Ztrans) (330). After the transition impedance (330) is reached, the level of impedance rises dramatically over time, and beyond this point the tissue impedance is generally too high for electrosurgical energy to have a substantial impact on the tissue. Therefore, termination (340) of the electrosurgical energy generally occurs soon after the transition impedance (330) point is reached. Thus, the period of time between the initial impedance (310) and when the transition impedance (330) is reached is generally the only effective time when electrosurgical energy may be applied to the tissue with any positive effect. This region is sometimes known as the bathtub region (350), due to the general shape of the curve over this time period. It is therefore desirable to extend or prolong the bathtub region (350) in order to increase the amount of time where electrosurgical energy may be applied.

### IV. Use of Composite Load Curves

Surgical system (98) may be programmed to provide power to a tissue bite between jaws (182, 184) according to any suitable method or algorithm. For example, in some instances, generator (200) may provide an electrosurgical drive signal according to one or more power curves. A power curve may define a relationship between power delivered to the tissue and the impedance of the tissue. For example, as the impedance of the tissue changes (e.g., increases) during coagulation, the power provided by generator (200) may also change (e.g., decrease) according to the applied power curve.

Different power curves may be particularly suited, or ill-suited, to different types and/or sizes of tissue bites. Aggressive power curves (e.g., power curves calling for high power levels) may be suited for large tissue bites. When applied to smaller tissue bites, such as small vessels, more aggressive power curves may lead to exterior searing or other deleterious effects. Exterior searing may reduce the coagulation/weld quality at the exterior and can also prevent complete coagulation of interior portions of the tissue. Similarly, less aggressive power curves may fail to achieve hemostasis when applied to larger tissue bites (e.g., larger bundles).

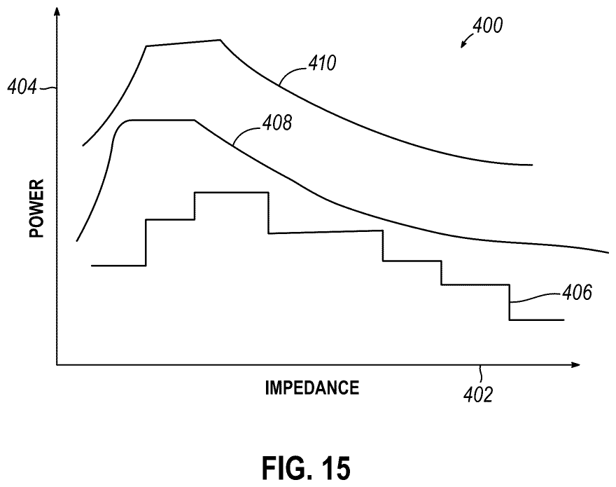

FIG. 15 shows an example of a chart (400) showing example power curves (406, 408, 410). Chart (400) comprises an impedance axis (402) showing increasing potential tissue impedances from left to right. A power axis (404) shows increasing power from down to up. Each of power curves (406, 408, 410) may define a set of power levels, on power axis (404), corresponding to a plurality of potential sensed tissue impedances, in impedance axis (402). In general, power curves may take different shapes, and this is illustrated in FIG. 15. Power curve (406) is shown with a step-wise shape, while power curves (408, 410) are shown with curved shapes. It will be appreciated that power curves utilized by various examples may take any usable continuous or non-continuous shape. The rate of power delivery or aggressiveness of a power curve may be indicated by its position on chart (400). For example, power curves that deliver higher power for a given tissue impedance may be considered more aggressive. Accordingly, between two power curves, the curve positioned highest on power axis (404) may be the more aggressive. It will be appreciated that some power curves may overlap.

It will be appreciated that during the coagulation or welding process, tissue impedance may generally increase. In some cases, tissue impedance may display a sudden impedance increase indicating successful coagulation. The increase may be due to physiological changes in the tissue, etc. The amount of energy that may be required to bring about the sudden impedance increase may be related to the thermal mass of the tissue being acted upon. The thermal mass of any given tissue bite, in turn, may be related to the type and amount of tissue in the bite.

This sudden increase in tissue impedance may be utilized to select an appropriate power curve for a given tissue bite. For example, generator (200) may select and apply successively more aggressive power curves until the tissue impedance reaches an impedance threshold indicating that the sudden increase has occurred. For example, reaching the impedance threshold may indicate that coagulation is progressing appropriately with the currently applied power curve. The impedance threshold may be a tissue impedance value, a rate of change of tissue impedance, and/or a combination of impedance and rate of change. For example, the impedance threshold may be met when a certain impedance value and/or rate of change are observed. According to various examples, different power curves may have different impedance thresholds, as described herein.

In some versions utilizing a pulsed drive signal, generator (200) may apply one or more composite load curves to the drive signal, and ultimately to the tissue. Composite load curves, like other power curves described herein, may define a level of power to be delivered to the tissue as a function of a measured tissue property or properties. Composite load curves may, additionally, define pulse characteristics, such as pulse width, in terms of the measured tissue properties (e.g., impedance, applied current, applied voltage, temperature, reflectivity, force applied to the tissue, etc.).

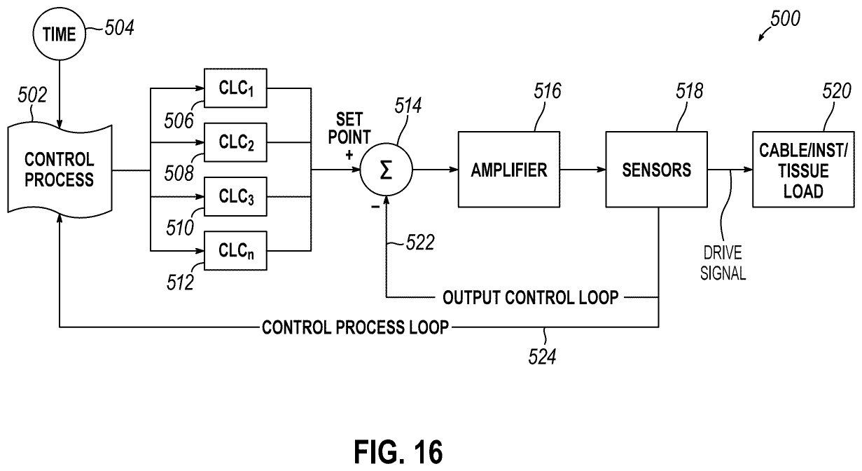

FIG. 16 illustrates one aspect of a logic diagram (500) for the selection and application of composite load curves by generator (200). It will be appreciated that logic diagram (500) may be implemented with any suitable type of generator or surgical device. According to various aspects, logic diagram (500) may be implemented utilizing an electrosurgical instrument, such as electrosurgical instrument (100) described above with respect to FIG. 1.

Referring back to FIG. 16, a control process (502) may be executed, for example by a digital device of generator (200) to select and apply composite load curves (506, 508, 510, 512). Control process (502) may receive a time input from a clock (504) and may also receive loop input (524) from sensors (518). Loop input (524) may represent properties or characteristics of the tissue that may be utilized in control process (502) to select and/or apply a composite load curve. Examples of such characteristics may comprise, for example, current, voltage, temperature, reflectivity, force delivered to the tissue, resonant frequency, rate of change of resonant frequency, etc. Sensors (518) may be dedicated sensors (e.g., thermometers, pressure sensors, etc.) or may be software implemented sensors for deriving tissue characteristics based on other system values (e.g., for observing and/or calculating voltage, current, tissue temperature, etc., based on the drive signal). Control process (502) may select one of the composite load curves (506, 508, 510, 512) to apply, for example based on loop input (524) and/or the time input from clock (504). Although four composite load curves are shown, it will be appreciated that any suitable number of composite load curves may be used.

Control process (502) may apply a selected composite load curve in any suitable manner. For example, control process (502) may use the selected composite load curve to calculate a power level and one or more pulse characteristics based on tissue impedance (e.g., currently measured tissue impedance may be a part of, or may be derived from, the loop input) or resonant frequency characteristics of an ultrasonic surgical instrument (100). Examples of pulse characteristics that may be determined based on tissue impedance according to a composite load curve may include pulse width, ramp time, and off time.

At set point (514), the derived power and pulse characteristics may be delivered to the drive signal. In various aspects, a feedback loop (522) may be implemented to allow for more accurate modulation of the drive signal. At the output of set point (514), the drive signal may be provided to an amplifier (516), which may provide suitable amplification. The amplified drive signal may be provided to a load (520) (e.g., via sensors (518)). Load (520) may comprise the tissue, surgical instrument (100), and/or any cable electrically coupling generator (200) with surgical instrument (100) (e.g., cable (10)).

V. Method of Applying Energy with Bathtub Region Exit Detection and Seal Completeness Sensing

In some instances, it may be desirable to provide reliable detection of the bathtub region exit. For example, it may be desirable to provide detection of the bathtub region exit that accounts for scenarios where the monitored tissue impedance might reach the first predetermined impedance threshold prior to the bathtub region exit, as may be the case when a vessel exhibits “vessel collapse” such that the monitored tissue impedance may rise as if the bathtub region exit is occurring, but may then drop back down before the bathtub region exit truly occurs. It may also be desirable to provide detection of the bathtub region that is not dependent on a minimum time threshold, so that the procedure can more quickly proceed to the next stage of the seal cycle for the post-bathtub region once the bathtub region exit has truly occurred. It will be appreciated that increased confidence that the bathtub region exit has truly occurred may allow for a decrease in risk mitigation that might otherwise be performed during the post-bathtub region, such as by obviating any need for a minimum time threshold for the duration of the post-bathtub region (since such a minimum time threshold may be premised on ensuring that the seal has truly exited the bathtub region and will hold pressure).

In addition, or alternatively, it may be desirable to provide reliable sensing of the seal completion. For example, it may be desirable to provide sensing of the seal completion that is not dependent on a minimum time threshold for the duration of the post-bathtub region, so that the seal cycle time can be reduced in cases where the minimum time threshold might otherwise be unnecessarily lengthy (e.g., for small vessels). It may also be desirable to provide increased confidence that the tissue is adequately sealed in cases where the minimum time threshold might otherwise be insufficient (e.g., for large vessels and bundles).

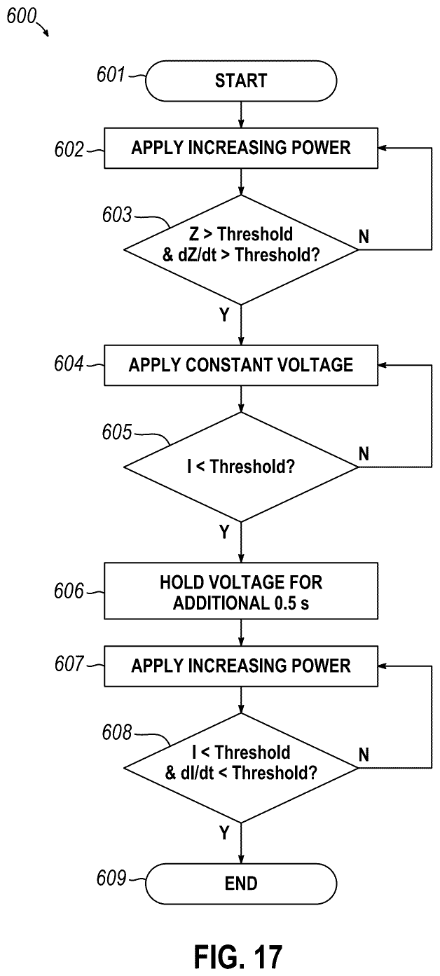

FIG. 17 shows an example of a tissue sealing method (600) that may provide one or more such functionalities. Method (600) begins at step (601), at which one or more electrodes of an electrosurgical instrument, such as electrodes (194, 196) of instrument (100), are positioned relative to target tissue to apply radio frequency (RF) energy to the target tissue, such as for sealing, coagulating, and/or welding the target tissue. For example, the target tissue may be clamped between jaws (182, 184) of end effector (180) of instrument (100), such that electrodes (194, 196) engage (e.g., contact) the target tissue. Step (601) may also include initiating energy delivery to the target tissue. Method (600) proceeds from step (601) to step (602), at which increasing power is applied to the target tissue, such as via electrodes (194, 196).

Method (600) proceeds from step (602) to step (603), at which the monitored tissue impedance (Z) is compared to a predetermined impedance threshold, and at which the monitored tissue impedance slope (dZ/dt) is compared to a predetermined impedance slope threshold, such as via the processor of control unit (102) or a processor incorporated into generator (200). In this regard, the tissue impedance (Z) may be monitored by the processor based on intermittent sensing of the impedance (Z) of the target tissue via electrodes (194, 196) in the manner described above; and the tissue impedance slope (dZ/dt) may be calculated by the processor based on the sensed tissue impedance (Z) and the time elapsed since the initiation of energy delivery. In the example shown, the processor queries at step (603) whether the monitored tissue impedance (Z) is greater than the predetermined impedance threshold, and also queries whether the monitored tissue impedance slope (dZ/dt) is greater than the predetermined impedance slope threshold.

If at step (603) the monitored tissue impedance (Z) is not greater than the predetermined impedance threshold, and/or if the monitored tissue impedance slope (dZ/dt) is not greater than the predetermined impedance slope threshold, then the processor may determine that the bathtub region exit has not yet occurred. Thus, in response to the monitored tissue impedance (Z) not being greater than the predetermined impedance threshold, and/or in response to the monitored tissue impedance slope (dZ/dt) not being greater than the predetermined impedance slope threshold, method (600) returns from step (603) to step (602) for continued application of increasing power to the target tissue.

If at step (603) the monitored tissue impedance (Z) is greater than the predetermined impedance threshold and the monitored tissue impedance slope (dZ/dt) is greater than the predetermined impedance slope threshold, then the processor may determine that the bathtub region exit has occurred. Thus, the processor may determine that the bathtub region exit has occurred, and may allow method (600) to proceed to the next stage for the post-bathtub region, without imposing any minimum time threshold. In the present example, in response to the monitored tissue impedance (Z) being greater than the predetermined impedance threshold and the monitored tissue impedance slope (dZ/dt) being greater than the predetermined impedance slope threshold, method (600) proceeds from step (603) to step (604), at which a predetermined constant voltage is applied to the target tissue, such as via electrodes (194, 196).

Method (600) proceeds from step (604) to step (605), at which the monitored current (I) is compared to a predetermined current threshold. In this regard, the current (I) applied to the target tissue may be monitored by the processor in any suitable manner, such as described above. In the example shown, the processor queries at step (605) whether the monitored current (I) is less than the predetermined current threshold.

If at step (605) the monitored current (I) is not less than the predetermined current threshold, then the processor may determine that instrument (100) has a continued ability to effectively deliver energy to the target tissue. Thus, in response to the monitored current (I) not being less than the predetermined current threshold, method (600) returns from step (605) to step (604) for continued application of the predetermined constant voltage to the target tissue.

If at step (605) the monitored current (I) is less than the predetermined current threshold, then the processor may determine that instrument (100) has a reduced ability to effectively deliver energy to the target tissue. In the present example, in response to the monitored current (I) being less than the predetermined current threshold, method (600) proceeds from step (605) to step (606), at which the application of the predetermined constant voltage to the target tissue is continued for a predetermined duration of time.

As shown, method (600) proceeds from step (606) to step (607), at which increasing power is applied to the target tissue, such as via electrodes (194, 196). The application of increasing power to the target tissue at step (607) may be referred to as a termination pulse.

Method (600) proceeds from step (607) to step (608), at which the monitored current (I) is compared to a predetermined current threshold, and at which the monitored current slope (dI/dt) is compared to a predetermined current slope threshold, such as via the processor. In this regard, the current (I) applied to the target tissue may be monitored by the processor in any suitable manner, such as described above; and the current slope (dI/dt) may be calculated by the processor based on the applied current (I) and the time elapsed since the initiation of energy delivery. In the example shown, the processor queries at step (608) whether the monitored current (I) is less than the predetermined current threshold, and also queries whether the monitored current slope (dI/dt) is less than the predetermined current slope threshold. In some cases, the predetermined current threshold applied at step (608) may be the same as that applied at step (605). In addition, or alternatively, the predetermined current slope threshold may be about 0 A/s, for example.

If at step (608) the monitored current (I) is not less than the predetermined current threshold, and/or if the monitored current slope (dI/dt) is not less than the predetermined current slope threshold, then the processor may determine that instrument (100) is still in the process of putting a final “sear” on the seal. Thus, in response to the monitored current (I) not being less than the predetermined current threshold, and/or in response to the monitored current slope (dI/dt) not being less than the predetermined current slope threshold, method (600) returns from step (608) to step (607) for continued application of increasing power to the target tissue.

If at step (608) the monitored current (I) is less than the predetermined current threshold and the monitored current slope (dI/dt) is less than the predetermined current slope threshold, then the processor may determine that a minimal amount of energy is being effectively delivered to the target tissue. Thus, the processor may determine that the target tissue has been adequately sealed, coagulated, and/or welded, without imposing a minimum time threshold (e.g., other than the predetermined duration of time applied at step (606)). In the present example, in response to the monitored current (I) being less than the predetermined current threshold and the monitored current slope (dI/dt) being less than the predetermined current slope threshold, method (600) proceeds from step (608) to step (609), at which energy delivery to the target tissue is terminated.

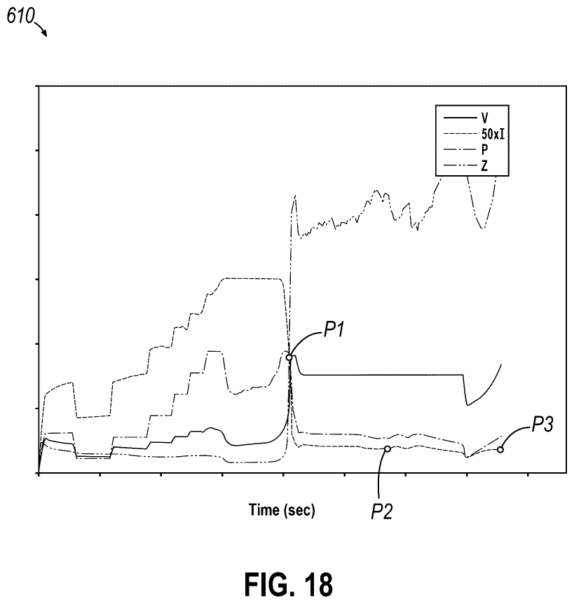

FIG. 18 shows an example of a graph (610) depicting relationships of each of the applied voltage, applied current, applied power, and tissue impedance with time that may occur when using method (600).

At plot point (P1) in graph (610), the monitored tissue impedance (Z) is greater than the predetermined impedance threshold and the monitored tissue impedance slope (dZ/dt) is greater than the predetermined impedance slope threshold. Thus, plot point (P1) may correspond to a determination by the processor at step (603) that the bathtub region exit has occurred, such that the predetermined constant voltage may then be applied to the target tissue per step (604).

At plot point (P2) in graph (610), the monitored current (I) is less than the predetermined current threshold. Thus, plot point (P2) may correspond to a determination by the processor at step (605) that instrument (100) has a reduced ability to effectively deliver energy to the target tissue, such that the application of the predetermined constant voltage to the target tissue may be continued for the predetermined duration of time per step (606), followed by applying increasing power to the target tissue per step (607).

At plot point (P3) in graph (610), the monitored current (I) is less than the predetermined current threshold and the monitored current slope (dI/dt) is less than the predetermined current slope threshold. Thus, plot point (P3) may correspond to a determination by the processor at step (608) that a minimal amount of energy is being effectively delivered to the target tissue, such that energy delivery to the target tissue may then be terminated per step (609).

### VI. Method of Applying Energy Based on Composite Load Curve Tables

In some instances, it may be desirable to allow for polynomial control of energy output and switching between different energy output levels without having the energy output level temporarily drop to zero, which might otherwise interrupt the sealing process. In addition, or alternatively, it may be desirable to allow the type of target tissue to be taken into account for determining appropriate energy delivery parameters suitable for the particular type of target tissue.

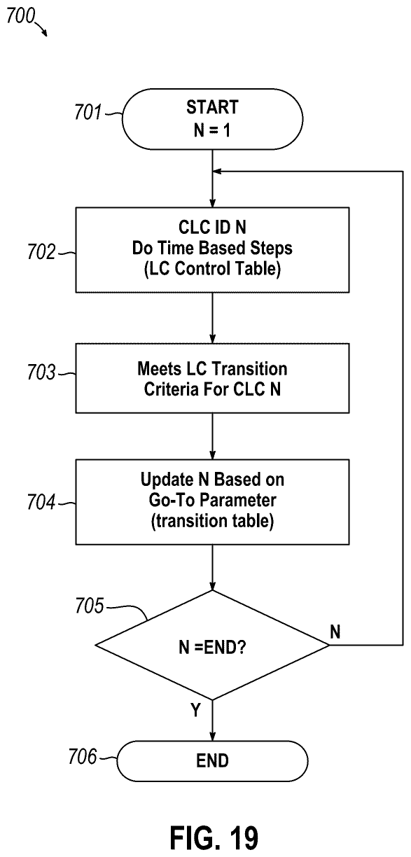

FIG. 19 shows an example of a tissue sealing method (700) that may provide one or more such functionalities. Method (700) begins at step (701), at which one or more electrodes of an electrosurgical instrument, such as electrodes (194, 196) of instrument (100), are positioned relative to target tissue to apply radio frequency (RF) energy to the target tissue, such as for sealing, coagulating, and/or welding the target tissue. For example, the target tissue may be clamped between jaws (182, 184) of end effector (180) of instrument (100), such that electrodes (194, 196) engage (e.g., contact) the target tissue. Step (701) may also include setting the value of N to 1. Method (700) proceeds from step (701) to step (702), at which time-based steps for a composite load curve having identification code N are performed based on a load curve control table. For example, when the value of N is set to 1, the time-based steps for a composite load curve having identification code 1 are performed based on the load curve control table. Such time-based steps may include energy delivery to the target tissue. Method (700) proceeds from step (702) to step (703), at which the load curve transition criteria for the composite load curve having identification code N are met. Method (700) proceeds from step (703) to step (704), at which the value of N is updated based on a load curve transition table. Method (700) proceeds from step (704) to step (705), at which a determination is made as to whether the current value of N is equal to an end value of N. If at step (705) the current value of N is not equal to the end value of N, then method (700) returns from step (705) to step (702) for performance of time-based steps for a composite load curve having identification code N based on a load curve control table. If at step (705) the current value of N is equal to the end value of N, then method (700) proceeds from step (705) to step (706), at which energy delivery to the target tissue is terminated.

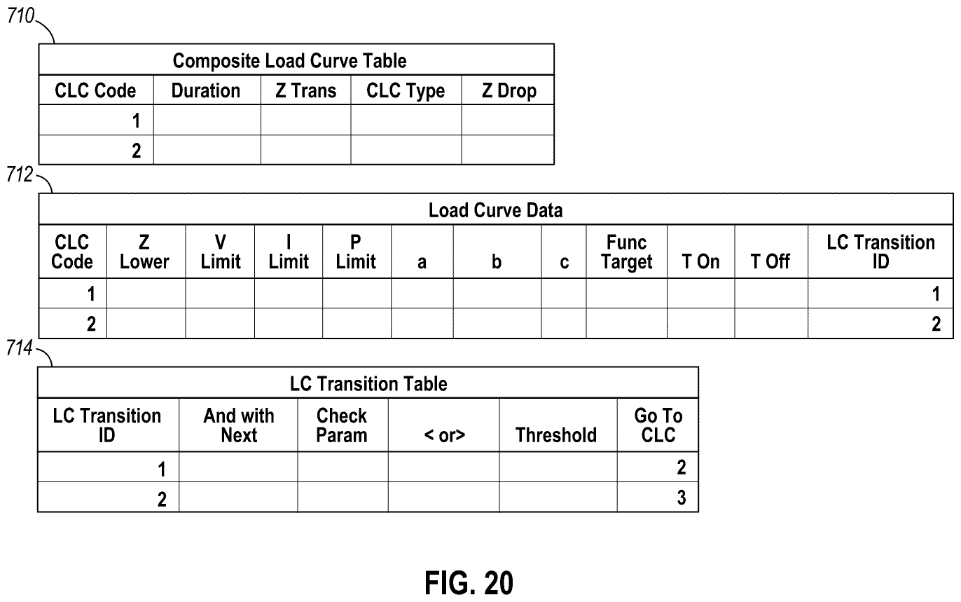

FIG. 20 shows examples of lookup tables (710, 712, 714) that may be applied during method (700). In some versions, lookup tables (710, 712, 714) may be stored on the memory unit of control unit (102), for example, and/or on a non-volatile memory unit (e.g., an EEPROM chip device) on instrument (100). Lookup tables (710, 712, 714) may be accessed by a suitable processor for executing method (700). For example, lookup tables (710, 712, 714) may be accessed by the processor of control unit (102) and/or by the processor of generator (200). In the example shown, lookup tables (710, 712, 714) include a composite load curve table (710), a load curve control table (712), and a load curve transition table (714). While various cells of lookup tables (710, 712, 714) are shown as being vacant, it will be appreciated that each cell may be populated with corresponding predetermined data as set forth below.

Composite load curve table (710) of the present example includes composite load curve data for composite load curves having identification codes 1 and 2, including respective duration values, impedance transition values, composite load curve types, and impedance drop (hysteresis) values. It will be appreciated that composite load curve table (710) may include composite load curve data for any suitable number of composite load curves (e.g., having identification codes 3, 4, 5, 6, etc.). In some cases, the duration value for each composite load curve may be either a fixed duration value, a maximum duration value, or a minimum duration value, based on the composite load curve type. The impedance transition value for each composite load curve may be the transition impedance level (Ztrans) (330) corresponding to the bathtub region exit for the respective composite load curve (see FIG. 14).

In some cases, the composite load curve type for each composite load curve may be selected from a value of 1, a value of 2, or a value of 3. For example, a value of 1 may indicate a composite load curve having a fixed duration (e.g., indicated by the respective duration value), such that the composite load curve may terminate based on the fixed duration elapsing; a value of 2 may indicate a composite load curve having a maximum duration (e.g., indicated by the respective duration value), such that the composite load curve may terminate based on either the maximum duration elapsing or another threshold criteria being satisfied (e.g., whichever happens first); and a value of 3 may indicate a composite load curve having a minimum duration (e.g., indicated by the respective duration value), such that the composite load curve may terminate based on both the minimum duration elapsing and another threshold criteria being satisfied (e.g., both conditions must be met). It will be appreciated that the composite load curves may have the same composite load curve type as each other, or may have different composite load curve types from each other, as may be desired. In some cases, composite load curve table (710) may include at least one of each of the above three types of composite load curves. The impedance drop (hysteresis) value for each composite load curve may be equal to another threshold criteria (e.g., a threshold impedance value) to be satisfied for termination of the composite load curve.

Load curve control table (712) of the present example includes load curve control data for composite load curves having identification codes 1 and 2, including respective lower impedance values, voltage limit values, current limit values, power limit values, a values, b values, c values, functional target identifiers, time on values, time off values, and load curve transition identification codes. It will be appreciated that load curve control table (712) may include load curve control data for any suitable number of composite load curves (e.g., having identification codes 3, 4, 5, 6, etc.). The lower impedance value for each composite load curve may be zero, for example. In some cases, the composite load curves may have any one or more of the same voltage limit value as each other; the same current limit value as each other; and/or the same current limit value as each other. For example, the composite load curves may have the same voltage limit value as each other; the same current limit value as each other; and the same current limit value as each other.

The a value, b value, and c value for each composite load curve indicate the constants in a polynomial function that is to be applied to either the voltage output, current output, or power output based on corresponding functional target identifier. For example, the polynomial function for each composite load curve may be expressed as:

$${at}^{2} + bt + c$$

where:
a=the corresponding a value;
b=the corresponding b value;
c=the corresponding c value; and
t=time.

In this regard, the functional target identifier for each composite load curve may be selected from an identifier of V, an identifier of I, or an identifier of P. For example, an identifier of V may indicate that the functional target for the composite load curve is the voltage output; an identifier of I may indicate that the functional target for the composite load curve is the current output; and an identifier of P may indicate that the functional target for the composite load curve is the power output. In some cases, the composite load curves may have any one or more of the same a value as each other; the same b value as each other; the same c value as each other; and/or the same functional target identifier as each other. For example, the composite load curves may have the same a value as each other; the same c value as each other; and the same functional target identifier as each other; but may have different b values from each other.

The functional target for each composite load curve follows the above polynomial function based on the functional target identifier, while the corresponding voltage, current, or power limit provides a maximum value for the function. For example, if the functional target identifier is V, the voltage limit value is 60 V, the a value is 0, the b value is 20, and the c value is 20, then the voltage may linearly ramp from 20 V to 60 V from 0 s to 2 s, and may then remain at 60 V for the remainder of the time; the other two outputs that are not the functional target (current and power in this example) may also be constrained by the respective limit values.

The time on value for each composite load curve may be the same as the corresponding duration value in composite load curve table (710). The time off value for each composite load curve may be zero, for example. In some cases, the load curve transition identification code for each composite load curve may be the same as the composite load curve identification code.

Load curve transition table (714) of the present example includes load curve transition data for load curve transitions having identification codes 1 and 2, including respective “and with next” identifier, “check param” (e.g., parameter to check) identifier, “< or >” (e.g., comparison) identifier, threshold value, and “Go To CLC” parameter. It will be appreciated that load curve transition table (714) may include load curve transition data for any suitable number of load curve transitions (e.g., having identification codes 3, 4, 5, 6, etc.). The “and with next” identifier for each load curve transition may be selected from an identifier of zero, an identifier of AND, or an identifier of OR. For example, an identifier of zero may indicate that the load curve transition is based on a single row of data in load curve transition table (714); an identifier of AND may indicate that the load curve transition is based on a combination of the current row of data with the next row of data in load curve transition table (714), using the “and” Boolean operator; and an identifier of OR may indicate that the load curve transition is based on a combination of the current row of data with the next row of data in load curve transition table (714), using the “or” Boolean operator.

The “check param” identifier for each load curve transition may be selected from an identifier of Z, an identifier of dZ/dt, an identifier of I, or an identifier of dI/dt. For example, an identifier of Z may indicate that the parameter to be checked is impedance; an identifier of dZ/dt may indicate that the parameter to be checked is impedance slope; an identifier of I may indicate that the parameter to be checked is current; and an identifier of dI/dt may indicate that the parameter to be checked is current slope. The “< or >” identifier for each load curve transition may be selected from an identifier of < or an identifier of >. For example, an identifier of < may indicate that the corresponding threshold value is a maximum threshold value; and an identifier of > may indicate that the corresponding threshold value is a minimum threshold value. In some cases, the threshold value for each load curve transition may be equal to the impedance drop (hysteresis) value for the corresponding composite load curve, such as where the “check param” identifier is Z and the “< or >” identifier is >. The “Go To CLC” parameter for each load curve transition may be selected from the composite load curve identification codes in composite load curve table (710) and load curve control table (712), or an end value. For example, a parameter of 1 may indicate transitioning to the composite load curve having identification code 1; a parameter of 2 may indicate transitioning to the composite load curve having identification code 2; and a parameter of an end value, such as 3 (e.g., where there is no composite load curve having an identification code 3 in composite load curve table (710) and load curve control table (712)), may indicate terminating energy delivery to the target tissue.

As noted above, lookup tables (710, 712, 714) may be applied during method (700). For example, with the value of N set to 1 per step (701), energy may be delivered to the target tissue in accordance with the composite load curve having identification code 1 via the time-based steps as indicated by load curve control table (712) per step (702). Once the load curve transition criteria for the composite load curve having identification code 1 have been satisfied as indicated by load curve transition table (714) per step (703), the value of N may be updated as indicated by the corresponding “Go To CLC” parameter of load curve transition table (714) per step (704). Per step (705), if the updated value of N is not an end value, then energy may be delivered to the target tissue in accordance with the composite load curve having identification code N via the time-based steps as indicated by load curve control table (712) per step (702); and if the updated value of N is an end value, then energy delivery to the target tissue may be terminated per step (706).

Thus, performing method (700) with lookup tables (710, 712, 714) may enable transitions between composite load curves and/or termination to be made based on any one or more of a duration of time, a magnitude of impedance, a slope of impedance over time, a magnitude of current, and/or a slope of current over time. The transition and/or termination conditions can be either a minimum or maximum value, and/or may be any combination of the above factors (e.g., as conditions that must all be satisfied, or as conditions that may be satisfied in the alternative). For example, as noted above, the bathtub region exit may be reliably detected based on the slope of impedance over time; and the minimal amount of energy that can be effectively delivered to the target tissue as the tissue approaches a complete seal can be reliably detected based on the magnitude of current as well as the slope of the current over time. Such reliable detection of the various tissue states may allow for an overall reduction in seal times.

Performing method (700) with lookup tables (710, 712, 714) may also enable transitioning between composite load curves in a nonlinear manner. For example, if a load curve transition is based on multiple transition conditions being satisfied, then the satisfaction of each transition condition can lead to different steps. This may allow for looping and/or branching within method (700). Looping (e.g., via the “Go To CLC” parameters of load curve transition table (714)) may allow for repeating a portion of method (700), such as in instances where an initial threshold condition (e.g., current) has been satisfied but a subsequent threshold condition (e.g., current slope) is not satisfied, such that reapplying a previous composite load curve may be desired. Branching (e.g., via the “Go To CLC” parameters of load curve transition table (714)) may allow for tissue classification, such that different power output and/or logic may be applied based on the particular type of target tissue.

The structure of lookup tables (710, 712, 714) may allow for smooth transitions between different levels of energy delivery. For example, if a transition from voltage 1 (V1) to voltage 2 (V2) to voltage 3 (V3) is desired, using lookup tables (710, 712, 714) may allow for generator (200) to transition directly from V1 to V2 to V3, without dropping to zero energy output in between V1 and V2 or in between V2 and V3; and/or may allow for generator (200) to apply energy with a linear or quadratic function that fits the transition between V1, V2, and V3. Avoiding drops to zero energy output and/or large jumps between energy output levels can help to avoid corresponding shocks to the target tissue, which can otherwise have unpredictable effects on the target tissue and/or interfere with the seal cycle.

### VII. Method of Applying Energy Based on Automated Tissue Classification

In some instances, it may be desirable to provide automated tissue classification of the target tissue. For example, the ability to differentiate between tissue types may allow for the application of different power output and/or logic (e.g., termination condition(s)) based on the particular type of target tissue, which may improve hemostasis performance, reduce seal times, and/or reduce thermal damage.

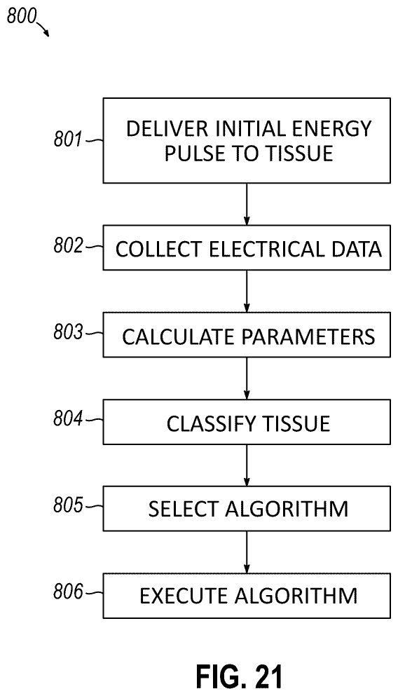

FIG. 21 shows an example of a tissue sealing method (800) that may provide one or more such functionalities. Method (800) begins at step (801), at which an initial energy pulse of a predetermined magnitude is delivered to the target tissue over a predetermined period of time, such as via electrodes (194, 196) of instrument (100). For example, the target tissue may be clamped between jaws (182, 184) of end effector (180) of instrument (100), such that electrodes (194, 196) engage (e.g., contact) the target tissue, and a nose pulse of 30 W may be delivered to the target tissue over a period of 0.25 s at step (801). Method (800) proceeds from step (801) to step (802), at which electrical data is collected for the predetermined period of time. Such electrical data may include any one or more of voltage, current, and impedance data.

Method (800) proceeds from step (802) to step (803), at which various parameters are calculated based on the electrical data collected at step (802), such as via the processor of control unit (102) or the processor of generator (200). Such parameters may include any one or more of the initial impedance, the initial current, the initial voltage, the final impedance, the final current, the final voltage, the minimum impedance, the minimum current, the minimum voltage, the maximum impedance, the maximum current, the maximum voltage, the average impedance slope, the average current slope, and/or the average voltage slope. In some cases, all of these parameters may be calculated. In some other cases, only some of these parameters may be calculated, such as only the initial impedance, the average impedance slope, and the average current slope.

Method (800) proceeds from step (803) to step (804), at which the type of target tissue is classified based on the parameters calculated at step (803). For example, all of the calculated parameters may be inputted into a support vector machine (SVM) or other suitable type of machine learning model that is configured to classify the type of target tissue based on the calculated parameters in real time. In some cases, the type of target tissue may be classified as an artery, vein, fat, thick avascular tissue (e.g., small intestine), or thin avascular tissue (e.g., mesometrium). As another example, such as in cases where only the initial impedance, the average impedance slope, and the average current slope are calculated, simple thresholds may be applied to the calculated parameters to determine if the target tissue is fat, an artery, a bundle, or avascular tissue, for example.

Method (800) proceeds from step (804) to step (805), at which an algorithm for energy delivery is selected based on the classification of the target tissue performed at step (804). For example, an algorithm for delivering energy to an artery may be selected based on the target tissue being classified as an artery at step (804). Method (800) proceeds from step (805) to step (806), at which the algorithm selected as step (805) is executed to deliver energy to the target tissue accordingly.

### VIII. Illustrative Combinations

The following examples relate to various non-exhaustive ways in which the teachings herein may be combined or applied. The following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A surgical system comprising: (a) a surgical instrument comprising an end effector, the end effector including: (i) a first jaw, (ii) a second jaw configured to cooperate with the first jaw to clamp tissue, and (iii) a plurality of electrodes configured to deliver radiofrequency (RF) energy to the tissue; and (b) a processor configured to: (i) control delivery of the RF energy to the tissue via the plurality of electrodes with increasing power, (ii) monitor an impedance of the tissue, (iii) calculate an impedance slope of the tissue over time, (iv) compare the impedance of the tissue to a predetermined impedance threshold, (v) compare the impedance slope of the tissue over time to a predetermined impedance slope threshold, (vi) in response to determining that the impedance of the tissue is not greater than the predetermined impedance threshold or that the impedance slope of the tissue over time is not greater than the predetermined impedance slope threshold, control the delivery of the RF energy to continue with increasing power, and (vii) in response to determining that the impedance of the tissue is greater than the predetermined impedance threshold and that the impedance slope of the tissue over time is greater than the predetermined impedance slope threshold, control the delivery of the RF energy with a predetermined constant voltage.

### Example 2

The surgical system of Example 1, wherein the processor is configured to monitor a current associated with the delivery of the RF energy with the predetermined constant voltage.

### Example 3

The surgical system of Example 2, wherein the processor is configured to compare the current to a predetermined current threshold.

### Example 4

The surgical system of Example 3, wherein the processor is configured to, in response to determining that the current is not less than the predetermined current threshold, control the delivery of the RF energy to continue with the predetermined constant voltage.

### Example 5

The surgical system of any of Examples 3 through 4, wherein the processor is configured to, in response to determining that the current is less than the predetermined current threshold: (i) control the delivery of the RF energy to continue with the predetermined constant voltage for a predetermined duration of time, and (ii) in response to the predetermined duration of time elapsing, control the delivery of the RF energy with increasing power.

### Example 6

The surgical system of Example 5, wherein the processor is configured to monitor a current associated with the delivery of the RF energy with increasing power.

### Example 7

The surgical system of Example 6, wherein the processor is configured to calculate a current slope over time.

### Example 8

The surgical system of Example 7, wherein the processor is configured to: (i) compare the current to a predetermined current threshold; and (ii) compare the current slope over time to a predetermined current slope threshold.

### Example 9

The surgical system of Example 8, wherein the processor is configured to, in response to determining that the current is not less than the predetermined current threshold or that the current slope over time is not less than the predetermined current slope threshold, control the delivery of the RF energy to continue with increasing power.

### Example 10

The surgical system of any of Examples 8 through 9, wherein the processor is configured to, in response to determining that the current is less than the predetermined current threshold and that the current slope over time is less than the predetermined current slope threshold, control the delivery of the RF energy to terminate.

### Example 11

The surgical system of any of Examples 1 through 10, wherein the processor is incorporated into the surgical instrument.

### Example 12

The surgical system of any of Examples 1 through 10, further comprising an RF energy generator, wherein the processor is incorporated into the RF energy generator.

### Example 13

The surgical system of any of Examples 1 through 12, wherein the processor is configured to control the delivery of the RF energy based on at least one load curve data lookup table.

### Example 14

The surgical system of Example 13, wherein the at least one load curve data lookup table is stored on a non-volatile memory unit on the surgical instrument.

### Example 15

The surgical system of any of Examples 1 through 14, wherein the processor is configured to: (i) deliver an initial energy pulse to the tissue via the plurality of electrodes, (ii) collect electrical data associated with the initial energy pulse, (iii) calculate electrical parameters based on the electrical data, (iv) determine a classification of the tissue based on the electrical parameters, and (v) control the delivery of the RF energy based on the classification of the tissue.

### Example 16

A method comprising: (a) clamping tissue between first and second jaws of an end effector such that a plurality of electrodes of the end effector contact the tissue; (b) delivering radiofrequency (RF) energy to the tissue via the plurality of electrodes with increasing power; (c) monitoring an impedance of the tissue; (d) calculating an impedance slope of the tissue over time; (e) comparing the impedance of the tissue to a predetermined impedance threshold; (f) comparing the impedance slope of the tissue over time to a predetermined impedance slope threshold; (g) in response to determining that the impedance of the tissue is not greater than the predetermined impedance threshold or that the impedance slope of the tissue over time is not greater than the predetermined impedance slope threshold, continuing to deliver the RF energy with increasing power; and (h) in response to determining that the impedance of the tissue is greater than the predetermined impedance threshold and that the impedance slope of the tissue over time is greater than the predetermined impedance slope threshold, delivering the RF energy with a predetermined constant voltage.

### Example 17

The method of Example 16, further comprising: (a) monitoring a current associated with delivering the RF energy with the predetermined constant voltage; (b) comparing the current to a predetermined current threshold; (c) in response to determining that the current is not less than the predetermined current threshold, continuing to deliver the RF energy with the predetermined constant voltage; (d) in response to determining that the current is less than the predetermined current threshold: (i) continuing to deliver the RF energy with the predetermined constant voltage for a predetermined duration of time, and (ii) in response to the predetermined duration of time elapsing, delivering the RF energy with increasing power.

### Example 18

The method of Example 17, further comprising: (a) monitoring a current associated with delivering the RF energy with increasing power; (b) calculating a current slope over time; (c) comparing the current to a predetermined current threshold; (d) comparing the current slope over time to a predetermined current slope threshold; (e) in response to determining that the current is not less than the predetermined current threshold or that the current slope over time is not less than the predetermined current slope threshold, continuing to deliver the RF energy with increasing power; and (f) in response to determining that the current is less than the predetermined current threshold and that the current slope over time is less than the predetermined current slope threshold, ceasing to deliver the RF energy.

### Example 19

The method of any of Examples 16 through 18, wherein the RF energy is delivered based on at least one load curve data lookup table.

### Example 20

The method of any of Examples 16 through 19, further comprising: (a) delivering an initial energy pulse to the tissue via the plurality of electrodes; (b) collecting electrical data associated with the initial energy pulse; (c) calculating electrical parameters based on the electrical data; and (d) determining a classification of the tissue based on the electrical parameters, wherein the RF energy is delivered based on the classification of the tissue.

### Example 21

A surgical system comprising: (a) a surgical instrument comprising an end effector, the end effector including: (i) a first jaw, (ii) a second jaw configured to cooperate with the first jaw to clamp tissue, and (iii) a plurality of electrodes configured to deliver radiofrequency (RF) energy to the tissue; and (b) a processor configured to: (i) control delivery of the RF energy to the tissue via the plurality of electrodes with a predetermined constant voltage, (ii) monitor a current associated with the delivery of the RF energy with the predetermined constant voltage, (iii) compare the current to a predetermined current threshold, (iv) in response to determining that the current is not less than the predetermined current threshold, control the delivery of the RF energy to continue with the predetermined constant voltage, and (v) in response to determining that the current is less than the predetermined current threshold: (A) control the delivery of the RF energy to continue with the predetermined constant voltage for a predetermined duration of time, and (B) in response to the predetermined duration of time elapsing, control the delivery of the RF energy with increasing power.

### Example 22

The surgical system of Example 21, wherein the processor is configured to monitor a current associated with the delivery of the RF energy with increasing power.

### Example 23

The surgical system of Example 22, wherein the processor is configured to calculate a current slope over time.

### Example 24

The surgical system of Example 23, wherein the processor is configured to: (i) compare the current to a predetermined current threshold; and (ii) compare the current slope over time to a predetermined current slope threshold.

### Example 25

The surgical system of Example 24, wherein the processor is configured to, in response to determining that the current is not less than the predetermined current threshold or that the current slope over time is not less than the predetermined current slope threshold, control the delivery of the RF energy to continue with increasing power.

### Example 26

The surgical system of any of Examples 24 through 25, wherein the processor is configured to, in response to determining that the current is less than the predetermined current threshold and that the current slope over time is less than the predetermined current slope threshold, control the delivery of the RF energy to terminate.

### Example 27

The surgical system of any of Examples 21 through 26, wherein the processor is configured to, prior to controlling the delivery of the RF energy with the predetermined constant voltage: (i) control the delivery of the RF energy with increasing power, (ii) monitor an impedance of the tissue, and (iii) calculate an impedance slope of the tissue over time.

### Example 28

The surgical system of Example 27, wherein the processor is configured to: (i) compare the impedance of the tissue to a predetermined impedance threshold, and (ii) compare the impedance slope of the tissue over time to a predetermined impedance slope threshold.

### Example 29

The surgical system of Example 28, wherein the processor is configured to, in response to determining that the impedance of the tissue is not greater than the predetermined impedance threshold or that the impedance slope of the tissue over time is not greater than the predetermined impedance slope threshold, control the delivery of the RF energy to continue with increasing power.

### Example 30

The surgical system of any of Examples 28 through 29, wherein the processor is configured to, in response to determining that the impedance of the tissue is greater than the predetermined impedance threshold and that the impedance slope of the tissue over time is greater than the predetermined impedance slope threshold, control the delivery of the RF energy with the predetermined constant voltage.

### Example 31

The surgical system of any of Examples 21 through 30, wherein the processor is incorporated into the surgical instrument.

### Example 32

The surgical system of any of Examples 21 through 30, further comprising an RF energy generator, wherein the processor is incorporated into the RF energy generator.

### Example 33

The surgical system of any of Examples 21 through 32, wherein the processor is configured to control the delivery of the RF energy based on at least one load curve data lookup table.

### Example 34

The surgical system of Example 33, wherein the at least one load curve data lookup table is stored on a non-volatile memory unit on the surgical instrument.

### Example 35

The surgical system of any of Examples 21 through 34, wherein the processor is configured to: (i) deliver an initial energy pulse to the tissue via the plurality of electrodes, (ii) collect electrical data associated with the initial energy pulse, (iii) calculate electrical parameters based on the electrical data, (iv) determine a classification of the tissue based on the electrical parameters, and (v) control the delivery of the RF energy based on the classification of the tissue.

### Example 36

A method comprising: (a) clamping tissue between first and second jaws of an end effector such that a plurality of electrodes of the end effector contact the tissue; (b) delivering radiofrequency (RF) energy to the tissue via the plurality of electrodes with a predetermined constant voltage; (c) monitoring a current associated with delivering the RF energy with the predetermined constant voltage; (d) comparing the current to a predetermined current threshold; (e) in response to determining that the current is not less than the predetermined current threshold, continuing to deliver the RF energy with the predetermined constant voltage; and (f) in response to determining that the current is less than the predetermined current threshold: (i) continuing to deliver the RF energy with the predetermined constant voltage for a predetermined duration of time, and (ii) in response to the predetermined duration of time elapsing, delivering the RF energy with increasing power.

### Example 37

The method of Example 36, further comprising: (a) monitoring a current associated with delivering the RF energy with increasing power; (b) calculating a current slope over time; (c) comparing the current to a predetermined current threshold; (d) comparing the current slope over time to a predetermined current slope threshold; (e) in response to determining that the current is not less than the predetermined current threshold or that the current slope over time is not less than the predetermined current slope threshold, continuing to deliver the RF energy with increasing power; and (f) in response to determining that the current is less than the predetermined current threshold and that the current slope over time is less than the predetermined current slope threshold, ceasing to deliver the RF energy.

### Example 38

The method of any of Examples 36 through 37, further comprising, prior to delivering the RF energy with the predetermined constant voltage: (a) delivering the RF energy with increasing power; (b) monitoring an impedance of the tissue; (c) calculating an impedance slope of the tissue over time; (d) comparing the impedance of the tissue to a predetermined impedance threshold; (e) comparing the impedance slope of the tissue over time to a predetermined impedance slope threshold; (f) in response to determining that the impedance of the tissue is not greater than the predetermined impedance threshold or that the impedance slope of the tissue over time is not greater than the predetermined impedance slope threshold, continuing to deliver the RF energy with increasing power; and (g) in response to determining that the impedance of the tissue is greater than the predetermined impedance threshold and that the impedance slope of the tissue over time is greater than the predetermined impedance slope threshold, delivering the RF energy with the predetermined constant voltage.

### Example 39

The method of any of Examples 36 through 38, wherein the RF energy is delivered based on at least one load curve data lookup table.

### Example 40

The method of any of Examples 36 through 39, further comprising: (a) delivering an initial energy pulse to the tissue via the plurality of electrodes; (b) collecting electrical data associated with the initial energy pulse; (c) calculating electrical parameters based on the electrical data; and (d) determining a classification of the tissue based on the electrical parameters, wherein the RF energy is delivered based on the classification of the tissue.

### Example 41

A surgical system comprising: (a) a surgical instrument comprising an end effector, the end effector including: (i) a first jaw, (ii) a second jaw configured to cooperate with the first jaw to clamp tissue, and (iii) a plurality of electrodes configured to deliver radiofrequency (RF) energy to the tissue; and (b) a processor configured to: (i) control delivery of the RF energy to the tissue via the plurality of electrodes with increasing power, (ii) monitor a current associated with the delivery of the RF energy with increasing power, (iii) calculate a current slope over time, (iv) compare the current to a predetermined current threshold, (v) compare the current slope over time to a predetermined current slope threshold, (vi) in response to determining that the current is not less than the predetermined current threshold or that the current slope over time is not less than the predetermined current slope threshold, control the delivery of the RF energy to continue with increasing power, and (vii) in response to determining that the current is less than the predetermined current threshold and that the current slope over time is less than the predetermined current slope threshold, control the delivery of the RF energy to terminate.

### Example 42

The surgical system of Example 41, wherein the processor is configured to, prior to controlling the delivery of the RF energy with increasing power: (i) control the delivery of the RF energy with a predetermined constant voltage, (ii) monitor a current associated with the delivery of the RF energy with the predetermined constant voltage, and (iii) compare the current to a predetermined current threshold.

### Example 43

The surgical system of Example 42, wherein the processor is configured to, in response to determining that the current is not less than the predetermined current threshold, control the delivery of the RF energy to continue with the predetermined constant voltage.

### Example 44

The surgical system of any of Examples 42 through 43, wherein the processor is configured to, in response to determining that the current is less than the predetermined current threshold control the delivery of the RF energy to continue with the predetermined constant voltage for a predetermined duration of time.

### Example 45

The surgical system of Example 44, wherein the processor is configured to, in response to the predetermined duration of time elapsing, control the delivery of the RF energy with increasing power.

### Example 46

The surgical system of any of Examples 42 through 45, wherein the processor is configured to, prior to controlling the delivery of the RF energy with the predetermined constant voltage: (i) control the delivery of the RF energy with increasing power, and (ii) monitor an impedance of the tissue.

### Example 47

The surgical system of Example 46, wherein the processor is configured to calculate an impedance slope of the tissue over time.

### Example 48

The surgical system of Example 47, wherein the processor is configured to: (i) compare the impedance of the tissue to a predetermined impedance threshold, and (ii) compare the impedance slope of the tissue over time to a predetermined impedance slope threshold.

### Example 49

The surgical system of Example 48, wherein the processor is configured to, in response to determining that the impedance of the tissue is not greater than the predetermined impedance threshold or that the impedance slope of the tissue over time is not greater than the predetermined impedance slope threshold, control the delivery of the RF energy to continue with increasing power.

### Example 50

The surgical system of any of Examples 48 through 49, wherein the processor is configured to, in response to determining that the impedance of the tissue is greater than the predetermined impedance threshold and that the impedance slope of the tissue over time is greater than the predetermined impedance slope threshold, control the delivery of the RF energy with the predetermined constant voltage.

### Example 51

The surgical system of any of Examples 41 through 50, wherein the processor is incorporated into the surgical instrument.

### Example 52

The surgical system of any of Examples 41 through 50, further comprising an RF energy generator, wherein the processor is incorporated into the RF energy generator.

### Example 53

The surgical system of any of Examples 41 through 52, wherein the processor is configured to control the delivery of the RF energy based on at least one load curve data lookup table.

### Example 54

The surgical system of Example 53, wherein the at least one load curve data lookup table is stored on a non-volatile memory unit on the surgical instrument.

### Example 55

The surgical system of any of Examples 41 through 54, wherein the processor is configured to: (i) deliver an initial energy pulse to the tissue via the plurality of electrodes, (ii) collect electrical data associated with the initial energy pulse, (iii) calculate electrical parameters based on the electrical data, (iv) determine a classification of the tissue based on the electrical parameters, and (v) control the delivery of the RF energy based on the classification of the tissue.

### Example 56

A method comprising: (a) clamping tissue between first and second jaws of an end effector such that a plurality of electrodes of the end effector contact the tissue; (b) delivering radiofrequency (RF) energy to the tissue via the plurality of electrodes with increasing power; (c) monitoring a current associated with delivering the RF energy with increasing power; (d) calculating a current slope over time; (e) comparing the current to a predetermined current threshold; (f) comparing the current slope over time to a predetermined current slope threshold; (g) in response to determining that the current is not less than the predetermined current threshold or that the current slope over time is not less than the predetermined current slope threshold, continuing to deliver the RF energy with increasing power, and (h) in response to determining that the current is less than the predetermined current threshold and that the current slope over time is less than the predetermined current slope threshold, ceasing to deliver the RF energy.

### Example 57

The method of Example 56, further comprising, prior to delivering the RF energy with increasing power: (a) delivering the RF energy with a predetermined constant voltage; (b) monitoring a current associated with delivering the RF energy with the predetermined constant voltage; (c) comparing the current to a predetermined current threshold; (d) in response to determining that the current is not less than the predetermined current threshold, continuing to deliver the RF energy with the predetermined constant voltage; and (e) in response to determining that the current is less than the predetermined current threshold: (i) continuing to deliver the RF energy with the predetermined constant voltage for a predetermined duration of time, and (ii) in response to the predetermined duration of time elapsing, delivering the RF energy with increasing power.

### Example 58

The method of Example 57, further comprising, prior to delivering the RF energy with the predetermined constant voltage: (a) delivering the RF energy with increasing power; (b) monitoring an impedance of the tissue; (c) calculating an impedance slope of the tissue over time; (d) comparing the impedance of the tissue to a predetermined impedance threshold; (e) comparing the impedance slope of the tissue over time to a predetermined impedance slope threshold; (f) in response to determining that the impedance of the tissue is not greater than the predetermined impedance threshold or that the impedance slope of the tissue over time is not greater than the predetermined impedance slope threshold, continuing to deliver the RF energy with increasing power; and (g) in response to determining that the impedance of the tissue is greater than the predetermined impedance threshold and that the impedance slope of the tissue over time is greater than the predetermined impedance slope threshold, delivering the RF energy with the predetermined constant voltage.

### Example 59

The method of any of Examples 56 through 58, wherein the RF energy is delivered based on at least one load curve data lookup table.

### Example 60

The method of any of Examples 56 through 59, further comprising: (a) delivering an initial energy pulse to the tissue via the plurality of electrodes; (b) collecting electrical data associated with the initial energy pulse; (c) calculating electrical parameters based on the electrical data; and (d) determining a classification of the tissue based on the electrical parameters, wherein the RF energy is delivered based on the classification of the tissue.

### IX. Miscellaneous

Any one or more of the teaching, expressions, embodiments, examples, etc. described herein may be combined with any one or more of the teachings, expressions, embodiments, examples, etc. described in U.S. patent application Ser. No. ______ [Atty. Ref. No. END9573USNP1], entitled “Electrosurgical Instrument with Impedance Spectroscopy and Method of Monitoring State of Instrument Jaws and Tissue,” filed on even date herewith; U.S. patent application Ser. No. ______ [Atty. Ref. No. END9574USNP1], entitled “Electrosurgical Instrument with Jaw Status Monitoring and Method of Adjusting Energy Activation,” filed on even date herewith; U.S. patent application Ser. No. ______ [Atty. Ref. No. END9575USNP1], entitled “Electrosurgical Instrument and Method of Monitoring Clamp Position to Adjust Energy Application,” filed on even date herewith; U.S. patent application Ser. No. ______ [Atty. Ref. No. END9576USNP1], entitled “Electrosurgical Instrument and Method of Detecting Tissue Accumulation on End Effector,” filed on even date herewith; and/or U.S. patent application Ser. No. ______ [Atty. Ref. No. END9609USNP1], entitled “Electrosurgical Instrument and Method of Frequency Monitoring for Sealing Tissue,” filed on even date herewith. The disclosure of each of these applications is incorporated by reference herein.

It should be understood that any of the versions of the instruments described herein may include various other features in addition to or in lieu of those described above. By way of example only, any of the devices herein may also include one or more of the various features disclosed in any of the various references that are incorporated by reference herein. Various suitable ways in which such teachings may be combined will be apparent to those of ordinary skill in the art.

While the examples herein are described mainly in the context of electrosurgical instruments, it should be understood that various teachings herein may be readily applied to a variety of other types of devices. By way of example only, the various teachings herein may be readily applied to other types of electrosurgical instruments, tissue graspers, tissue retrieval pouch deploying instruments, surgical staplers, surgical clip appliers, ultrasonic surgical instruments, etc. It should also be understood that the teachings herein may be readily applied to any of the instruments described in any of the references cited herein, such that the teachings herein may be readily combined with the teachings of any of the references cited herein in numerous ways. Other types of instruments into which the teachings herein may be incorporated will be apparent to those of ordinary skill in the art.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions or other disclosure material set forth herein will only be incorporated to the extent that no conflict arises between that incorporated material and the existing disclosure material.

Versions of the devices described above may have application in conventional medical treatments and procedures conducted by a medical professional, as well as application in robotic-assisted medical treatments and procedures. By way of example only, various teachings herein may be readily incorporated into a robotic surgical system such as the DAVINCI™ system by Intuitive Surgical, Inc., of Sunnyvale, California. Similarly, those of ordinary skill in the art will recognize that various teachings herein may be readily combined with various teachings of U.S. Pat. No. 6,783,524, entitled “Robotic Surgical Tool with Ultrasound Cauterizing and Cutting Instrument,” published Aug. 31, 2004, the disclosure of which is incorporated by reference herein, in its entirety.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A surgical system comprising:
(a) a surgical instrument comprising an end effector, the end effector including:
(i) a first jaw,
(ii) a second jaw configured to cooperate with the first jaw to clamp tissue, and
(iii) a plurality of electrodes configured to deliver radiofrequency (RF) energy to the tissue; and
(b) a processor configured to:
(i) control delivery of the RF energy to the tissue via the plurality of electrodes with increasing power,
(ii) monitor an impedance of the tissue,
(iii) calculate an impedance slope of the tissue over time,
(iv) compare the impedance of the tissue to a predetermined impedance threshold,
(v) compare the impedance slope of the tissue over time to a predetermined impedance slope threshold,
(vi) in response to determining that the impedance of the tissue is not greater than the predetermined impedance threshold or that the impedance slope of the tissue over time is not greater than the predetermined impedance slope threshold, control the delivery of the RF energy to continue with increasing power, and
(vii) in response to determining that the impedance of the tissue is greater than the predetermined impedance threshold and that the impedance slope of the tissue over time is greater than the predetermined impedance slope threshold, control the delivery of the RF energy with a predetermined constant voltage.

2. The surgical system of claim 1, wherein the processor is configured to monitor a current associated with the delivery of the RF energy with the predetermined constant voltage.

3. The surgical system of claim 2, wherein the processor is configured to compare the current to a predetermined current threshold.

4. The surgical system of claim 3, wherein the processor is configured to, in response to determining that the current is not less than the predetermined current threshold, control the delivery of the RF energy to continue with the predetermined constant voltage.

5. The surgical system of claim 3, wherein the processor is configured to, in response to determining that the current is less than the predetermined current threshold:
(i) control the delivery of the RF energy to continue with the predetermined constant voltage for a predetermined duration of time, and
(ii) in response to the predetermined duration of time elapsing, control the delivery of the RF energy with increasing power.

6. The surgical system of claim 5, wherein the processor is configured to monitor a current associated with the delivery of the RF energy with increasing power.

7. The surgical system of claim 6, wherein the processor is configured to calculate a current slope over time.

8. The surgical system of claim 7, wherein the processor is configured to:
(i) compare the current to a predetermined current threshold; and
(ii) compare the current slope over time to a predetermined current slope threshold.

9. The surgical system of claim 8, wherein the processor is configured to, in response to determining that the current is not less than the predetermined current threshold or that the current slope over time is not less than the predetermined current slope threshold, control the delivery of the RF energy to continue with increasing power.

10. The surgical system of claim 8, wherein the processor is configured to, in response to determining that the current is less than the predetermined current threshold and that the current slope over time is less than the predetermined current slope threshold, control the delivery of the RF energy to terminate.

11. A method comprising:
(a) clamping tissue between first and second jaws of an end effector such that a plurality of electrodes of the end effector contact the tissue;
(b) delivering radiofrequency (RF) energy to the tissue via the plurality of electrodes with increasing power;
(c) monitoring an impedance of the tissue;
(d) calculating an impedance slope of the tissue over time;
(e) comparing the impedance of the tissue to a predetermined impedance threshold;
(f) comparing the impedance slope of the tissue over time to a predetermined impedance slope threshold;
(g) in response to determining that the impedance of the tissue is not greater than the predetermined impedance threshold or that the impedance slope of the tissue over time is not greater than the predetermined impedance slope threshold, continuing to deliver the RF energy with increasing power; and
(h) in response to determining that the impedance of the tissue is greater than the predetermined impedance threshold and that the impedance slope of the tissue over time is greater than the predetermined impedance slope threshold, delivering the RF energy with a predetermined constant voltage.

12. The method of claim 11, further comprising:
(a) monitoring a current associated with delivering the RF energy with the predetermined constant voltage;
(b) comparing the current to a predetermined current threshold;
(c) in response to determining that the current is not less than the predetermined current threshold, continuing to deliver the RF energy with the predetermined constant voltage;
(d) in response to determining that the current is less than the predetermined current threshold:
(i) continuing to deliver the RF energy with the predetermined constant voltage for a predetermined duration of time, and
(ii) in response to the predetermined duration of time elapsing, delivering the RF energy with increasing power.

13. The method of claim 12, further comprising:
(a) monitoring a current associated with delivering the RF energy with increasing power;
(b) calculating a current slope over time;
(c) comparing the current to a predetermined current threshold;
(d) comparing the current slope over time to a predetermined current slope threshold;
(e) in response to determining that the current is not less than the predetermined current threshold or that the current slope over time is not less than the predetermined current slope threshold, continuing to deliver the RF energy with increasing power; and
(f) in response to determining that the current is less than the predetermined current threshold and that the current slope over time is less than the predetermined current slope threshold, ceasing to deliver the RF energy.

14. The method of claim 11, wherein the RF energy is delivered based on at least one load curve data lookup table.

15. The method of any of claim 11, further comprising:
(a) delivering an initial energy pulse to the tissue via the plurality of electrodes;
(b) collecting electrical data associated with the initial energy pulse;
(c) calculating electrical parameters based on the electrical data; and
(d) determining a classification of the tissue based on the electrical parameters, wherein the RF energy is delivered based on the classification of the tissue.

16. A surgical system comprising:
(a) a surgical instrument comprising an end effector, the end effector including:
(i) a first jaw,
(ii) a second jaw configured to cooperate with the first jaw to clamp tissue, and
(iii) a plurality of electrodes configured to deliver radiofrequency (RF) energy to the tissue; and
(b) a processor configured to:
(i) control delivery of the RF energy to the tissue via the plurality of electrodes with a predetermined constant voltage,
(ii) monitor a current associated with the delivery of the RF energy with the predetermined constant voltage,
(iii) compare the current to a predetermined current threshold,
(iv) in response to determining that the current is not less than the predetermined current threshold, control the delivery of the RF energy to continue with the predetermined constant voltage, and
(v) in response to determining that the current is less than the predetermined current threshold:
(A) control the delivery of the RF energy to continue with the predetermined constant voltage for a predetermined duration of time, and
(B) in response to the predetermined duration of time elapsing, control the delivery of the RF energy with increasing power.

17. The surgical system of claim 16, wherein the processor is configured to monitor a current associated with the delivery of the RF energy with increasing power.

18. The surgical system of claim 17, wherein the processor is configured to calculate a current slope over time.

19. The surgical system of claim 18, wherein the processor is configured to:
(i) compare the current to a predetermined current threshold;
(ii) compare the current slope over time to a predetermined current slope threshold; and
(iii) in response to determining that the current is not less than the predetermined current threshold or that the current slope over time is not less than the predetermined current slope threshold, control the delivery of the RF energy to continue with increasing power.

20. The surgical system of claim 16, wherein the processor is configured to control the delivery of the RF energy based on at least one load curve data lookup table, wherein the at least one load curve data lookup table is stored on a non-volatile memory unit on the surgical instrument.
